(19) United States
(12) Patent Application Publication
(10) Pub. No.: US 2017/0367810 A1
(43) Pub. Date: Dec. 28, 2017

(54) DEVICES AND METHODS FOR LUNG VOLUME REDUCTION

(71) Applicant: SHIFAMED HOLDINGS, LLC, Campbell, CA (US)

(72) Inventors: Don TANAKA, Saratoga, CA (US); Tom SAUL, Moss Beach, CA (US); Thomas McGRATH, Santa Clara, CA (US); Jeffery A. KROLIK, Campbell, CA (US); Jeff BARBOUR, Campbell, CA (US); Amr SALAHIEH, Saratoga, CA (US); Lilip LAU, Los Altos, CA (US)

(21) Appl. No.: 15/536,012

(22) PCT Filed: Jan. 13, 2016
(86) PCT No.: PCT/US2016/013140

§ 371 Date: Jun. 14, 2017

Related U.S. Application Data
(60) Provisional application No. 62/103,509, filed on Jan. 14, 2015.

(51) Int. Cl. A61F 2/04; A61B 17/12; A61B 17/04

(52) U.S. Cl. CPC A61F 2/04; A61B 17/12104; A61B 17/12131; A61B 17/12013; A61B 2017/0427; A61B 2017/0464; A61B 2017/0417

(57) ABSTRACT

Methods, devices, and systems for mechanically reducing the volume of the lung. Some embodiments include endobronchially positioning an anchoring device within a lung, the anchoring device comprising at least a distal anchor, expanding at least a portion of the distal anchor to anchor the distal anchor against lung lumen tissue, and tensioning at least a portion of the device to reduce the volume of the lung.

### CROSS-REFERENCE TO RELATED APPLICATIONS

This application claims the priority of U.S. Provisional Application No. 62/103,509, filed Jan. 14, 2015, which is incorporated by reference herein.

This application incorporates by reference herein the following applications: U.S. Provisional Application No. 61/845,355, filed Jul. 11, 2013; U.S. Provisional Application No. 61/846,992, filed Jul. 16, 2013; U.S. Provisional Application No. 61/856,227, filed Jul. 19, 2013; U.S. Provisional Application No. 61/906,711, filed Nov. 20, 2013; U.S. Provisional Application No. 61/914,330, filed Dec. 10, 2013; U.S. Provisional Application No. 61/921,070, filed Dec. 26, 2013; U.S. Provisional Application No. 61/934,638, filed Jan. 31, 2014; and PCT/US2014/046410, filed Jul. 11, 2014.

This application incorporates by reference herein the disclosure of U.S. Provisional Application No. 61/938,352, filed Feb. 11, 2014.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE DISCLOSURE

Lung volume reduction (LVR) is an important procedure in the treatment of emphysema or chronic bronchitis, a form of Chronic Obstructive Pulmonary Disease (COPD). COPD is the third leading cause of death in the United States. Emphysema is a type of COPD involving damage to the air sacs (alveoli) in the lungs. As it worsens, emphysema turns the alveoli into large, irregular pockets with gaping holes in their inner walls. This reduces the surface area of the lungs and, in turn, the amount of oxygen that reaches the bloodstream during each breadth. The damaged lung tissue additionally loses its ability to hold its normal shape and becomes hyper-inflated, thereby consuming a larger volume than comparable healthy tissue. Emphysema also slowly destroys the elastic fibers that hold open the small airways leading to the air sacs. This allows these airways to collapse upon exhalation, trapping air in the lungs. Treatment may slow the progression of emphysema, but it can't reverse the damage. The disclosure described herein comprise minimally invasive treatments intended to bring relief to patients suffering from the stages of emphysema where diseased portions of the lung no longer efficiently contribute to the oxygenation of the blood, but instead provide a hindrance to lung function and capacity.

Emphysema is often classified as to how uniformly diseased tissue or how uniformly the diseased state of the tissue is distributed through the lung. The two extremes are heterogeneous, where there are distinct pockets of diseased tissue separated by healthier tissue, and homogeneous, where the distribution of the diseased state of the tissue is more uniform. When there is a heterogeneous presentation it is useful to reduce the volume of the most diseased area of a lung. When the presentation is homogeneous it is useful to treat a portion of the most diseased lobe of the lung.

### SUMMARY OF THE DISCLOSURE

The disclosure described herein relates to apparatuses and methods which provide for minimally invasive treatment via LVR in patients suffering from emphysema by providing mechanical compression of the emphysematous tissue. This compression serves to reduce the volume occupied by the emphysematous tissue. Additionally, the compression of diseased tissue restores some of the lost compliance or elasticity of the original tissue and allows for the distal airways to remain open during exhalation, thereby allowing the release of trapped gas from within the healthy tissue. This procedure provides the benefits of surgical lung volume reduction while minimizing the risks associated with the far more invasive surgical procedure.

The apparatus of this disclosure comprises an anchoring system which in turn comprises at least two anchors connected to one another by a tethering structure, the system configured such that the distance between the two anchors can be decreased. In some embodiments the two anchors are comprised of at least a proximal anchor, at least a distal anchor, the at least one distal anchor and the at least one proximal anchor connected to one another by a tether, and a mechanism to decrease the distance between the proximal and distal anchors. In some embodiments there will be more than one distal anchors connected to a proximal anchor. In an alternate embodiment, the two anchors will be distal anchors and the proximal anchor will be the interface between the tether and a bifurcation in the bronchi. In many embodiments the distal anchor will be a fixation anchor designed to affix to the surrounding tissue, typically the wall of an airway, and in some cases additionally the tissues surrounding the airway.

In some embodiments the distance between two anchors may be adjusted by shortening the tether, in others by reducing the amount of tether between the two anchors. For the purposes of discussions herein foreshortening will describe either means of reducing the distance between anchors spanned by a tether. In some preferred embodiments the distance between an at least one proximal and one or more distal anchor(s) is adjustable such that the distance may be increased, or decreased. Yet other anchor embodiments allow for the release of the tether completely. A number of embodiments described in which the tether is shortened follow. The proximal anchor comprises a way of twisting the tether on itself such that the tether winds on itself, thereby foreshortening. The tether comprises a spring which on deployment shortens. Some embodiment in which the distance between two anchors is reduced by reducing the length of tether between two anchors are as follows. The proximal anchor comprises a means of winding the tether onto a spool. The tether is pulled through a catch mechanism comprised in the anchor. Additionally the tether comprises a feature which interfaces with the catch mechanism. The tether is comprised of a material which can be caused to shrink, such as by denaturation resulting from heating or a pH change, after deployment. Twisting or spooling of the tether and thereby managing any and all excess tether length that may result from the tensioning and foreshortening of the tether on implementing a lung volume reduction reduces the likelihood of the anchoring system causing an inflammatory response within the lung. Once the volume of the lung is reduced in the desired area, the remaining portion of the lung continues to function. This dynamic motion could exacerbate any local damage or inflammatory response that excess tether or protruding features may cause.

As used herein a fixation anchor is a device which is designed to be affixed to an airway. Such anchors comprise a fixation mechanism which fixes the anchor to the airway wall. In some embodiments the fixation means is a mechanical means where fixation results from a mechanical interference with the airway wall. Mechanical embodiments may pierce the airway wall, rely on local expansion of the airway, rely on the branching characteristic of the airways, rely on the alveolar interface at the terminus of the airways. In alternate embodiments the fixation may be by adhesive means, and in others it use combinations of the above.

Some embodiments presented herein use a spike as fixation means. The spike is incorporated into the anchor such that, when deployed, tensions applied to the spike by the anchoring system, as a distal and proximal anchor are drawn together, will drive the spike into, and maintain the spike in, the airway wall. In such embodiments the spikes may be configured such that upon release form a delivery device the spikes will move from a delivery configuration, in which the spikes are directed at an angle roughly along the longitudinal axis of the anchor, to a delivered configuration in which the spikes are directed at least partially radially outward. In other embodiments the spikes may be maintained in the delivery configuration by a removable wire or tab which is removed at the time of deployment. Such embodiments comprise an actuable fixation means. In some embodiments the spikes may be barbed such that once the tip passes through the airway wall the barb inhibits the ability of the airway wall to slip off the spike. In yet other embodiments the distal fixation means may comprise the whole anchor. Such an embodiment is comprised in a tagging fastener where the end of the tether comprises the fixation anchor. In a tagging fastener the fixation anchor portion of the tether is “T” shaped. During deployment the top of the “T” is folded parallel to the stem of the “T” and is passed through the wall of an airway. After passing the end through the airway wall it relaxes into its deployed state where it takes the shape of the “T”. The top of the “T” now locking the tether to the airway. In some embodiments the tether may be terminated by a volume of porous material which is saturated by an adhesive delivered via a lumen in the tether.

In alternate embodiments the fixation means is purely mechanical in nature, where the airway wall is not breached by the fixation means. Such embodiments comprise any of the following. Expanding structures such as spiral springs which expand the airway wall to a point where the structure is unable to slip. An anchor comprised of an array of interconnected distal airways filled with an adhesive or expanding material such as a PMMA or a collagen plug.

In some embodiments each proximal anchor will connect to one distal anchor. In others, each proximal anchor will connect with one distal anchor. In yet other embodiments the anchoring features will be distributed along the entire extent of the anchoring structure.

In some embodiments the proximal anchors will be placed in tissue less diseased than that in which the distal anchors are placed. Such an embodiment will be particularly useful in treating a more heterogeneous presentation of the disease. In other embodiments the distal anchors will be placed in tissues at the borders of diseased tissue also useful in treating a more heterogeneous presentation. In other embodiments the anchors will be placed in airways surrounded by tissues of a relatively uniform disease state such as in a homogeneous presentation where the tissues of a particular lobe are of a relatively uniform diseased state, but the particular lobe is more diseased the other lobes of the lung.

In some embodiments of this disclosure the anchors will be drawn together in a sequential fashion. Such a sequential foreshortening minimizes stress gradients across the volume reduced tissue both during the procedure and after completion of the procedure thereby reducing the risk of tears arising in the tissue and resultant loss in the total volume reduction. In a sequential procedure multiple anchor systems and or anchors within an anchor system will be foreshortened in an incremental fashion. Each tether will be foreshortened incrementally by an amount less than the total expected for the end LVR. In this way each tether will be foreshortened multiple times during the procedure. Alternatively, sequential may mean foreshortening one tether at a time.

In some instances such as when treating heterogeneous emphysematous tissue where some anchors can be placed in the peripheral healthier tissue at the borders of the more diseased tissue, and others are placed within more diseased tissues, the sequential procedure will allow the peripheral anchors to be drawn up first followed by those in the less healthy tissue. In such a situation it can be desirable to draw in the boundary tissues more than the central anchors to start. As the healthier tissue compresses in on the less healthy tissue less force will be required to draw in the less healthy tissue thereby reducing the risks of tears in the tissue. In situations where the tissue is of more uniform quality, adjusting each anchor by a consistent amount and cycling through all of the anchors multiple times will be more advantageous. In any procedure if tears are observed either by imaging or other means to be described, the foreshortening of individual anchors can be reversed relieving the stress gradients across the tissue. In such situations additional anchors may also be placed. Such a procedure is facilitated when performed under Fluoro or other medical imaging system.

Prior to any procedure a pre-evaluation can be performed to facilitate the eventual procedure. Such a pre evaluation can comprise any of the following procedures. Imaging procedures such as CT, standard Xray, Fluoroscopy (Fluoro), MRI, or ultrasound. Functional evaluations such as FEV1, RV, FVC, TLC, or other lung function test. Additionally tests which provide insights into the compliance, both dynamic and static, and or density distribution of the lung tissue will be useful. For the purpose of characterizing density and compliance an intrabronchial ultrasound will be useful.

After the pre-procedure evaluations are concluded a planning step will be performed. Such a step may be performed at the time of the LVR procedure and incorporate additional evaluations or it may be performed prior to the LVR procedure. The planning step will comprise some combination of the following. The identification of regions to be treated based on, density and or compliance as determined by medical imaging. An intrabronchial ultrasound can be particularly useful in such determinations, especially when preformed during the procedure. The identification of boundary between emphysematous and normal tissue using any of the techniques described herein. A determination of the number of and location of devices to be placed within and around or at the boundary of the emphysematous tissue. A determination of an initial goal for amount of tissue reduction predicated on any of the evaluations described herein.

A stepwise reduction may be performed in addition to or in combination with sequential reduction. In a stepwise reduction a period of time is allowed to pass prior to each incremental reduction, where each incremental reduction may comprise a foreshortening of all tethers or some subset of all of the tethers. A stepwise reduction may comprise any combination of the following. A stepwise reduction predicated on a healing response. Such a procedure would incorporate some combination of the following steps. Implant a set of anchors then apply coordinated sequential loading, load or displacement, to each anchor. The target magnitude of the loading or displacement increments characterized by any of the evaluations performed previously or elsewhere herein. The amount of displacement or loading applied determined using flouro, force measurements or torque measurements. Allow for tissue stabilization for a period of 5 minutes to 3 months (or more such as out to one or more years) depending on the magnitude of the healing response desired. Repeat the process until the desired LVR is achieved.

Alternatively or in combination the stepwise procedure may be predicated on allowing for an initial ingrowth/fixation of the anchors. Such a procedure would comprise some combination of the following steps. Implant anchors and allow tissue ingrowth to stabilize for a period of 7 days to 3 months. Then apply coordinated sequential loading load or displacement to each anchor. The target magnitude of the loading or displacement increments characterized by any of the evaluations performed previously. The amount of displacement or loading applied determined using flouro, force measurements or torque measurements. Allow for tissue stabilization for a period of 5 minutes to 3 months (or more such as out to one or more years) depending on the magnitude of the healing response desired. Repeat the process until the desired LVR is achieved. The process can be repeated until the desired outcome is achieved. In some circumstances adjustments may be repeated at time periods of one year or more to accommodate further deterioration of the emphysematous condition.

In some embodiments the device is implanted but lung volume is not immediately reduced. This can be done to allow initial ingrowth/fixation as discussed herein with risk of tearing of tissue. Methods of reducing lung volume can therefor include endobronchially delivering an anchoring device to a location within the lung within a delivery device, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device, deploying the anchoring device completely out of the delivery device, and removing the delivery device from the lung without increasing or decreasing the distance between the proximal and distal anchors. After a period of time that has sufficiently allowed fixation or ingrowth, the lung volume is then reduced.

In stepwise and sequential procedures the number reductions can be predicated on the pre evaluation and or pre procedure data. Procedure planning and pre-characterization of tissue quality can improve procedure outcome and is an important part of such procedures.

Some of the procedures described herein are facilitated by apparatus comprising some combination of the following. A flexible multi-lumen catheter suitable for use in an airway. Catheters comprising balloons or multiple balloons which may be used as temporary or permanent anchoring devices. Balloons which are permeable and allow for an adhesive to permeate through the balloon wall. Medical grade tissue adhesives or bioadhesives for use in fixing anchoring components. Space filling bio-materials such as gels and solids such as epoxies. Catheters comprising a means for penetrating the airway wall such as a directable hypo-tube capable of piercing the wall of an airway and delivering a mechanical anchor to a target area, and or delivering an adhesive or space filling material to a target area. Catheters comprising optical means such as a flexible fiber-optic fiber or LED capable of light by which the adhesive may be cured and other means for curing adhesives and space filling materials. In some embodiments a flexible fiber-optic tube capable of delivering both a light-curable adhesive and the light by which the adhesive may be cured may be used. A flexible catheter and balloon system capable of delivering an adhesive and providing a specified vacuum force to a target area. Such systems capable of releasing the implant portions of any anchoring system.

Some of the apparatus may additionally comprise devices capable of performing diagnostics such as the following. An intra-bronchial ultrasound transducer for use in characterizing density or compliance of local tissue. Alternatively, electrodes may be provided to allow for electrical impedance (EI) measurements as a way of characterizing tissue electrical impedance as a function of hyper inflated state and or changes in tissue electrical impedance as a function of tissue compression arising from the lung volume reduction. In other embodiments electrical impedance changes between multiple anchors may be used to indicate appropriate compression or tearing of tissues between the multiple anchors. In these embodiments the methods can include endobronchially positioning a tissue characterizing device within the lung, activating the characterizing device at one or more locations in the lung, and endobronchially deploying a distal anchor of a lung volume reduction device within the lung at a target location after determining that the target location of the lung is emphysematous tissue.

To enhance the efficacy and safety of the sequential and stepwise foreshortening procedures anchors may have load monitoring means incorporated into their structure. Alternatively load may be derived from the amount of spiraled tether as noted by fluoroscopy. Alternatively the amount of torque required to foreshorten a tether will indicate the forces acting on the tether. In such systems the force to displacement behavior may be monitored to indicate how the tissue under volume reduction is responding. When tissue begins to tear as noted by a decrease in load associated with a foreshortening the user may back off and lengthen that tether thereby removing tension. Alternate surrounding tethers or new tethers can be placed in the surrounding tissues. Alternatively or in combination some form of stepwise procedure may be instituted. In some embodiments the force displacement curves are displayed real time to the user. In some embodiments the expected maximum compression of portions of the lung to be treated will be predicted by density and or compliance measurements and these predictions used to inform the size of load or displacement increments to be applied during a sequential tether foreshortening procedure.

In some circumstances, such as when the treatment in a non responder provides no or minimal clinically positive outcomes, it may be desired by the physician to return the patient to the pre-operative state, or as close as possible to it. Some embodiments include reducing the tension applied to the lung tissue. In other embodiments, the proximal anchor or the entering anchoring device can be removed.

One aspect of the disclosure is a device for reducing the volume of a lung, comprising: a distal anchor, a proximal anchor, and a tether extending between the distal and proximal anchors, the device configured so that the distance between the anchors measured along the tether can be increased or decreased and maintained after release of a delivery device.

In some embodiments of this aspect the device is further configured so that the distance between the anchors can be further increased or decreased after the device has been released from a delivery device.

In some embodiments of this aspect the device further comprises a tensioning controller that interfaces with the tether, the tensioning controller configured to be actuated to increase or decrease the distance between the proximal and distal anchors.

In some embodiments of this aspect a tether actual length between the anchors stays the same. The tether can be adapted to be reconfigured such that the distance measured along the tether between the anchors can be reduced. In some embodiments only a portion of the tether is configured to be reconfigured.

In some embodiments of this aspect the tether is configured to wind up on itself to decrease the distance between the anchors.

In some embodiments of this aspect the distal anchor is disposed at a distal end of the device, the proximal anchor disposed at a proximal end of the device, and the device does not include any other anchors disposed between the distal and proximal anchors.

In some embodiments of this aspect the distal and proximal anchors are expandable.

In some embodiments of this aspect at least one of the distal and proximal anchors has an electrode thereon.

In some embodiments of this aspect the device is configured so that as the distance between anchors changes, a tether axis remains in the same direction. The axis can remain in the same direction even though the tether changes configuration.

In some embodiments of this aspect the device is configured so that as the distance between anchors changes, the rotational orientation, out of a plane comprising the tether axis, of the distal anchor stays the same relative to the proximal anchor.

In some embodiments of this aspect the proximal anchor is configured to be collapsed and removed from the lung after it has been expanded towards an expanded configuration. The distal anchor can be configured to be collapsed and removed from the lung after it has been expanded towards an expanded configuration.

One aspect of the disclosure is a method of reducing the volume of a lung, comprising endobronchially deploying an anchoring device within the lung, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device; reducing the volume of the lung by decreasing the distance between the distal and proximal anchors; and maintaining the decreased distance.

In some embodiments of this aspect the method further comprises, after the positioning step, releasing the anchoring device from a delivery device and removing the delivery device from the lung without decreasing the distance between the proximal and distal anchors, wherein the reducing and maintaining steps are performed after the releasing and removing steps. The reducing and maintaining steps can be performed after a second delivery device is endobronchially positioned within the lung.

In some embodiments of this aspect, after the maintaining step, waiting a period of time during which the distance between the anchors is not changed, and after the waiting step, at least one of increasing or decreasing the distance between the proximal and distal anchors. The waiting step can comprise monitoring a characteristic of the lung. The waiting step can comprise waiting a period of time for at least one of the following to occur: tissue relaxation, tissue ingrowth into one or both anchors; and a healing response in the volume reduced tissue. The method can comprise, after the waiting step, decreasing the distance between the proximal and distal anchors to further reduce the volume of the lung. The waiting step can comprise waiting at least 2 minutes during which the distance between the anchors is not changed.

In some embodiments of this aspect decreasing the distance comprises increasing the tension in the tether.

In some embodiments of this aspect, after the maintaining step, increasing the tension in a second tether extending from a second distal anchor also positioned in the lung. Increasing the tension in a second tether can comprise increasing the tension in a second tether that is coupled to a second proximal anchor different than the proximal anchor. Increasing the tension in a second tether can comprise increasing the tension in a second tether that is coupled to the proximal anchor.

In some embodiments of this aspect the method further comprises endobronchially positioning a second anchoring device within the lung, the second anchoring device comprising a second distal anchor, a second proximal anchor, and a second tether extending between the second distal and second proximal anchors, the second device configured such that the distance between the second distal and second proximal anchors can be increased or decreased and then maintained after release of the second anchoring device from a delivery device.

In some embodiments of this aspect decreasing the distance comprises causing at least a portion of the tether to wind up on itself.

In some embodiments of this aspect the method further comprises, prior to the deploying step, characterizing a physical quality of lung tissue using an endobronchially placed characterization device. Characterizing a physical quality of a portion of the lung can comprise characterizing a physical quality of the lung that is indicative of emphysematous tissue. The physical quality can be at least one of tissue compliance and tissue density. After the characterizing step characterizes the portion of the lung as emphysematous tissue, the method can include anchoring the distal anchor in the emphysematous tissue. The characterizing step can comprise measuring the electrical impedance of the lung tissue. The method can also include determining a maximum tension to apply to the distal anchor using the results of the characterizing step.

In some embodiments of this aspect decreasing the distance between the distal and proximal anchors comprises actuating a tension controller secured to the proximal anchor.

In some embodiments of this aspect the method further comprises, after the reducing step, increasing the lung volume by adjusting the anchoring device. Adjusting the anchoring device can comprise increasing the distance between the anchors. Adjusting the anchoring device can comprise removing the proximal anchor from the lung. Adjusting the anchoring device can comprise removing the distal anchor from the lung.

One aspect of the disclosure is a method of reducing lung volume, comprising endobronchially positioning a tissue characterizing device within the lung; activating the characterizing device at one or more locations in the lung; and endobronchially deploying a distal anchor of a lung volume reduction device within the lung at a target location after determining that the target location of the lung is emphysematous tissue. The activating step comprises activating an electrical impedance device, wherein the distal anchor includes an electrode thereon. The activating step can comprise activating an electrical impedance device, wherein a delivery device includes an electrode thereon. The activating step can comprise activating an ultrasound device on a delivery tool.

One aspect of the disclosure is a method of reducing lung volume, comprising endobronchially reducing a volume of lung with a lung volume reduction device; waiting a period of time at least 2 minutes without further reducing the volume of the lung; and after the waiting step, further reducing the volume of the lung.

One aspect of the disclosure is a method of reducing lung volume, comprising endobronchially reducing a volume of lung with a lung volume reduction device; after the reducing step, waiting a period of time without further reducing lung volume sufficient to allow at least one of tissue relaxation, tissue ingrowth into a part of the device; and a healing response in the volume of reduced tissue to occur; and after the waiting step, further reducing the volume of the lung.

One aspect of the disclosure is a method of reducing the volume of a lung, comprising endobronchially delivering an anchoring device to a location within the lung within a delivery device, the anchoring device comprising a distal anchor, a proximal anchor, and a tether extending between the distal and proximal anchors, the device configured such that the distance between the distal and proximal anchors measured along the tether can be increased or decreased and then maintained after release of the anchoring device from a delivery device; deploying the anchoring device completely out of the delivery device; and removing the delivery device from the lung without increasing or decreasing the distance between the proximal and distal anchors.

### BRIEF DESCRIPTION OF THE DRAWINGS

FIGS. 1A-1C illustrates an exemplary treatment device comprised of three components.

FIG. 2 shows the airway anchor in a cutaway view.

FIG. 3 identifies structures of the lung for the purposes of simplification. Additionally, a portion of the parenchyma is afflicted with emphysema.

FIG. 4 shows a bronchoscope tracked into the airway leading to the emphysematous tissue to be treated.

FIG. 5 a distal anchor is deployed from the treatment device.

FIG. 6 the delivery sheath is withdrawn further back into the bronchoscope to deploy the proximal anchor.

FIG. 7 a drive shaft engages with the interface of a socket in the proximal anchor.

FIGS. 8A and 8B illustrate drive shaft rotation transmitted through the socket and into the tether, with the distal anchor drawn into closer proximity to the proximal anchor.

FIG. 9 a volumetric reduction in the emphysematous portion of the lung can be observed.

FIGS. 10 and 11 a preferred embodiment having a tension monitoring mechanism is shown.

FIGS. 12, 13A, 13B, 14A, 14B, 15, 16, 17, and 18 show a mechanism used to hold and adjust tension within a tether. This design allows for a completely adjustable and reversible tension to be applied to tethers within the airways of the lung.

FIG. 19 shows a top view of an embodiment of a tensioning mechanism, while

FIG. 20 shows the side view and FIG. 21 shows the front of the same design.

FIG. 22 shows a top view of another embodiment of a tensioning mechanism while

FIG. 23 shows the side view of the same design.

FIG. 24 shows a top view of another embodiment of a tensioning mechanism, while

FIG. 25 shows the side view of the same design.

FIG. 26 shows a top view of another embodiment of a tensioning mechanism while

FIG. 27 shows the side view of the same design.

FIG. 28 shows an alternative embodiment to that shown in FIGS. 29 and 30.

FIG. 29 shows a first embodiment of an adjustable anchor system for lung volume reduction.

FIG. 30 shows the first embodiment of the adjustable anchor system for lung volume reduction after the tethers have been tightened.

FIG. 31 is a section view of an emphysematous lung.

FIGS. 32 and 33 are an example of a single lung anchor utilized for lung volume reduction. Tension applied to the anchor in FIG. 33 has exceeded the tensile strength of the parenchyma resulting in a tear.

FIGS. 34 and 35 are an example of multiple lung anchors with applied tension loads spread over a larger area thereby avoiding tears in the surrounding tissue.

FIGS. 36A-36D illustrate different outcomes to the surrounding tissue based on the timing of applied tension to distal anchors during lung volume reduction.

FIG. 37 shows the top view of one embodiment of a tensioning mechanism for each tether;

FIG. 38 shows the side view of the same design.

FIG. 39 shows the top view of another embodiment of a tensioning mechanism;

FIG. 40 shows the side view of the same design.

FIG. 41 shows the top view of another embodiment of a tensioning mechanism, while

FIG. 42 shows the side view of the same design.

FIG. 43 shows the top view of another embodiment of a tensioning mechanism;

FIG. 44 shows the bottom view from FIG. 43.

FIG. 45 shows a side view of the same design from FIG. 43.

FIG. 46 shows a top view of tether and anchoring system, while

FIG. 47 shows a side view of the same design.

FIG. 48 shows a top view of tether and anchoring system, while

FIG. 49 shows a side view of the same design.

FIG. 50 a spring element design for the purpose of lung volume reduction is shown.

FIG. 51 device shape-set to have a relaxed state resembling that of a helix.

FIG. 52 device set to resemble a torsional spring.

FIG. 53 illustrates how a device with longitudinal element wrapping in a spiral axis and stretched would appear.

FIG. 54 shows a foreshortening for the helical design.

FIG. 55 illustrates that of the torsional spring configuration.

FIG. 56 the tissue along the airway wall surrounding the device is engaged and drawn together.

FIG. 57 and FIG. 58 show tissue engaged and the feature length simultaneously foreshortens reducing the length of the tissue of the airway wall.

FIG. 59, a series of sharp tines run along the trailing edge of the longitudinal element.

FIG. 60, tine feature can be incorporated into a raised element within the face of the element.

FIG. 61 shows another embodiment of a tensioning mechanism.

FIG. 62 shows another embodiment of a tensioning mechanism.

FIG. 63 shows another embodiment of a tensioning mechanism.

FIG. 64, hypothetical target for volume reduction shown in the upper right of the illustration.

FIG. 65, single device or multiple devices are individually introduced into the desired airway.

FIG. 66, device is released and foreshortening from the spring force it draws in the engaged tissue, compressing the volume of the tissue attached to the airway in that portion of the lung.

FIG. 67, devices can stand alone as a unitary feature, or can be connected to a central node.

FIGS. 68A and 68B show a flat pattern design for a stent-like anchor that could be delivered to the periphery of the airway in the lung.

FIG. 69 shows a lung with a diseased upper lobe. An endoscope has been tracked within the bronchial tree so that its tip is engaging within the upper lobe.

FIG. 70 shows a small diameter catheter is advanced into a segment of distal bronchial lumen, the catheter tip in a segment of the bronchial tree having one or more bifurcations within its structure.

FIG. 71, a curable material is injected into the segment of distal bronchial lumen.

FIG. 72, additional small diameter catheters are placed, and curable material injected and cured.

FIG. 73, an anchoring catheter is advanced as far forward as possible, stopping upon reaching a bifurcation of the bronchial tree.

FIG. 74, an anchor is deployed to stabilize the anchoring catheter in the bronchial tree.

FIG. 75, small diameter catheters are retracted back into the anchoring catheter.

FIG. 76, small diameter catheters are trimmed at the anchor, and the anchor is detached from the anchoring catheter.

FIG. 77, a primary anchor balloon is deployed with a smaller retractable balloon.

FIGS. 78A and 78B, an anchor balloon is established in an airway through which a catheter delivers flexible tubes capable of delivering an adhesive.

FIGS. 79A, 79B and 79C, a multi-lumen delivery catheter equipped to install multiple anchoring and adhesive points and to provide various treatment monitoring and feedback components is described.

FIG. 79D, an outer airway wall is pierced by a hypo-tube or hollow tether producing an adhesive bleb at the outer airway wall.

FIGS. 80A-80C illustrate two possible styles of barbed lead.

FIGS. 81A-81B employ a “T”-style barbed lead in an alternate anchoring system.

FIGS. 82A-82F and 83A-83B present another embodiment of the present disclosure incorporating the hypotube delivery of a looping stitch.

FIG. 84 shows a graph of the relationship between load applied to a distal anchor and the resulting displacement of that anchor.

FIG. 85 and FIG. 86 show the impact of emphysematous tissue on the relationship between load applied and the resulting displacement of an anchor.

FIG. 87 shows the relationship between torque applied to the line attached to anchors described in FIG. 85.

FIG. 88 shows the torque applied in a line attached between anchors as a function of the number of turns applied to that anchor.

FIG. 89A shows the line before it has reached its limit in number of turns before forming a loop.

FIG. 89B shows the line having loops in it after the line has increased past point “T” of FIG. 84.

FIG. 90 illustrates an exemplary anchoring device with electrodes disposed on the anchors.

FIG. 91 presents a flow chart of possible steps for use in performing a lung volume reduction as described herein.

FIG. 92A-F illustrate an exemplary sequence of using an exemplary device for reducing a volume of a lung.

FIGS. 93A-D illustrate an exemplary sequence of deploying an exemplary anchor.

FIGS. 94A-D illustrate an exemplary sequence of deploying an exemplary anchor.

FIGS. 95A-C illustrate an exemplary sequence of deploying an exemplary anchor.

FIGS. 96A-D illustrate an exemplary sequence of deploying an exemplary anchor.

FIGS. 97A-B illustrate an exemplary sequence of deploying an exemplary anchor.

FIG. 98 illustrates an exemplary anchor in an expanded configuration.

FIG. 99 illustrates an exemplary anchor in an expanded configuration.

FIGS. 100A-E illustrate an exemplary sequence of using an exemplary device for reducing a volume of a lung.

### DETAILED DESCRIPTION OF THE DISCLOSURE

The disclosure describes methods, devices, and systems for reducing the volume of a lung.

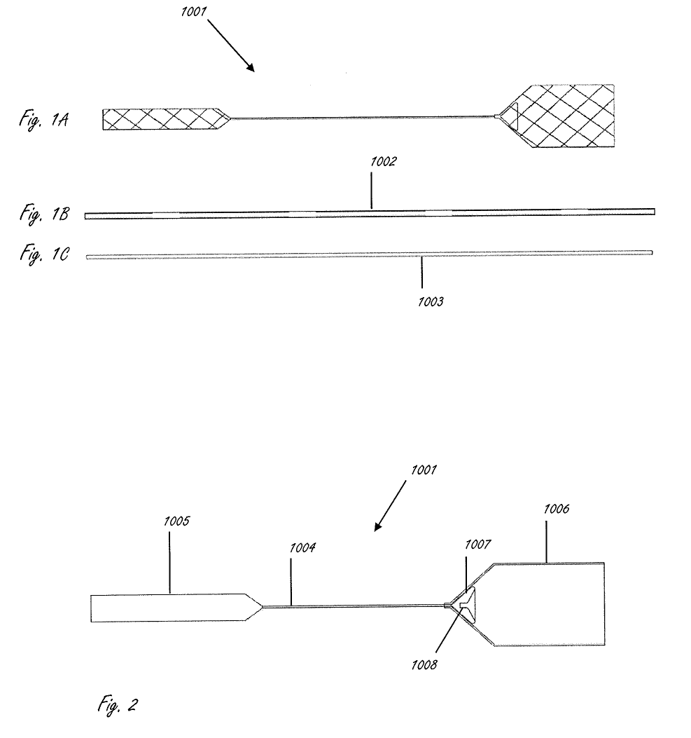

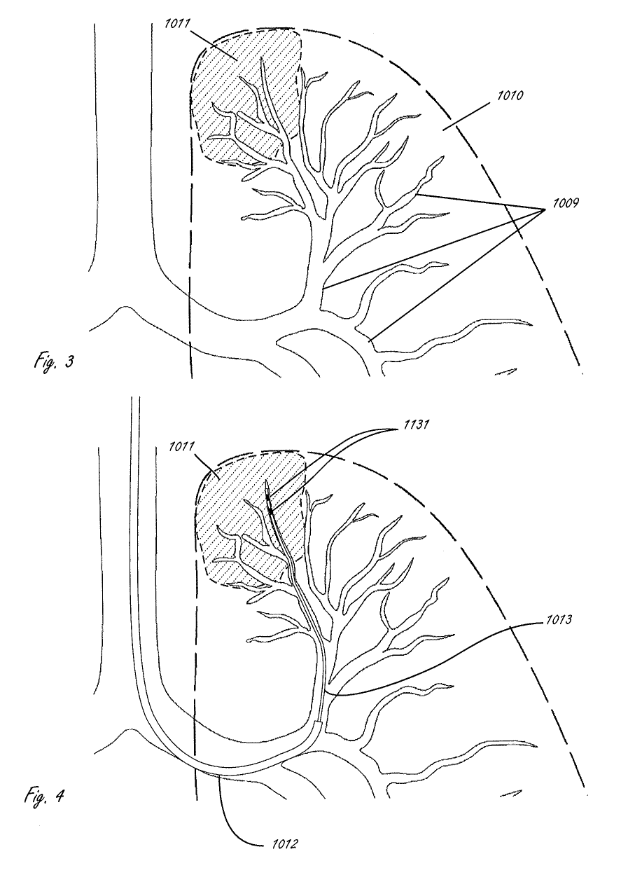

FIGS. 1A-1C and 2 illustrate an exemplary embodiment of a lung volume reduction apparatus. The embodiment in FIGS. 1A-1C and 2 is an example of a device for reducing the volume of a lung that includes a distal anchor, a proximal anchor, and a tether extending between the distal and proximal anchors, the device configured so that the distance between the anchors measured along the tether can be increased or decreased and maintained after release of a delivery device. Apparatuses and devices configured and/or adapted to reduce the volume of a lung may also be referred to herein as “treatment devices.” The apparatus shown in FIGS. 1A-1C includes three components. The first component is an airway anchor (1001) as shown in FIG. 1A. An “airway anchor” may also be referred to herein as an “airway anchoring device” or other derivative. The airway anchor is designed to be collapsed into a small profile and delivered by the second component, a delivery sheath (1002), which is illustrated in FIG. 1B. The third component of the apparatus is a drive shaft (1003), shown in FIG. 1C, configured to tighten the airway anchor (1001) once the airway anchor is positioned in its target location. The act of “tightening” as used herein may also be referred to herein as “tensioning.” Delivery sheath (1002) shown in FIG. 1B includes a lumen configured to house therein a plurality of separate anchoring devices, the plurality of anchoring devices positioned along the length of the lumen. That is, the anchoring devices are disposed within the lumen axially from one another rather than radially. In this embodiment an inner lumen of delivery sheath includes four anchor housing regions, each for receiving an anchor therein. The distal two regions thus receive the distal and proximal anchors of a first anchoring device, and the proximal two regions receive the distal and proximal anchors of a second anchoring device. The lumen can be configured to stably house any number of anchoring devices therein. The use of multiple anchoring devices is described below.

FIG. 2 illustrates a sectional view of airway anchor (1001) from FIG. 1A. The airway anchor (1001) includes an actuable distal anchor (1005), which is configured to be expanded from a first compressed configuration that allows it to be collapsed within delivery sheath (1002) for delivery to an expanded configuration for engaging an airway wall. Such exemplary expansible structures may include laser cut nitinol, braided nitinol, inflatable structures, and the like. The distal anchor (1005) may comprise a plurality of tines (as described further below) to maintain traction with the airway wall. The airway anchor (1001) also includes tether (1004) that is fixedly attached to the distal anchor (1005) on one end, and attached to, while maintaining rotation freedom from, the proximal anchor (1006). In this embodiment tether (1004) is constructed of material that maintains a high tensile and torsional strength to prevent breakage. In this embodiment tether (1004) is also somewhat flexible, so that upon twisting it is capable of winding itself into a non-straight configuration, and therefore becoming shorter without breaking or transmitting excessive torque to the distal anchor.

In some embodiments the tether is any of or a combination of Dacron®, Dyneema®, Spectra and Kevlar®. The tether can be a wide variety of common fishing line. In some embodiments braided Dacron® can be used. The tether can be a monofilament, a nanofilament (i.e., hundreds of longitudinal strands), as well as braided.

Adequate volume reduction may be achieved with reductions in proximal to distal anchor distance associated with less than a few percent of initial length, especially when initial tether length is great, and up to 100%, especially when initial tether lengths are short. Large reductions may be effected in multiple smaller increments with time periods allowed between reductions for tissue relaxation or healing as is described elsewhere herein. The effect of the sum of local anchor adjustments will typically support lobal lung volume reductions of up to 30%, more typically 20%, and some situations, such as but not limited to when tissue is particularly friable, less than 20% perhaps only a few percent. Local tissue volume reductions may be even greater.

In some embodiments the tether winds up on itself when twisted. In these embodiments the tether may wind up in a very controlled and repeatable configuration, or it may wind up and take on a variety of configurations. In either case the winding is reliable and repeatable, even if the wound up configuration is not completely predictable. In some embodiments the tether could be material used for fishing line, that when twisted will wind up, or bunch up, on itself.

The airway anchor (1001) also includes proximal anchor (1006). Similarly to the distal anchor (1005), proximal anchor (1006) is configured to be expansible from first compressed configuration so that it can fit within the delivery sheath (1002), to a larger expanded configuration for engaging the airway wall. Such expansible structures may include laser cut nitinol, braided nitinol, or inflatable structures and the like. The proximal anchor (1006) may optionally include a plurality of tines (as described further below) to maintain traction with the airway wall. The anchoring device also includes socket (1007), which is secured to the proximal anchor (1006), and which is mechanically connected to tether (1004), but allows the tether to rotate within and with respect to the proximal anchor. The socket (1007) includes an interface (1008) configured to receive drive shaft (1003) therein. The drive shaft and interface are configured such that the drive shaft, when positioned in the socket, is rotational fixed with respect to the socket. Rotation of the drive shaft thus causes rotation of the socket. This arrangement allows the user to engage the drive shaft (1003) into the socket (1007) of the proximal anchor (1006), and twist the tether by twisting the drive shaft. The act of twisting the tether changes the configuration of the tether from a straight configuration to a non-straight configuration, resulting in the distal and proximal anchors being drawn together, and the distance between the anchors measured along the tether reduced.

FIGS. 1A-1C and 2 illustrate a merely exemplary lung volume reduction device and additional exemplary devices are descried below. FIGS. 3-9 illustrate an exemplary method of using the device shown in FIGS. 1A-1C and 2.

FIG. 3 illustrates a portion of a lung, a complex organ composed of airways, blood vessels, alveolar tissue, lymphatic tissue among other structures. In this section, only major airways (1009) and parenchyma (1010) will be referred to for the purposes of simplification. Major airways (1009) refers to the bronchi that carry air to and from the parenchyma (1010) for oxygen transport. The parenchyma (1010) refers to all other structures in the lung, a majority volume of which is alveolar tissue. In FIG. 3, both major airways (1009) and parenchyma (1010) are present. Additionally, a portion of the parenchyma (1011) shown with grey shading is afflicted with emphysema.

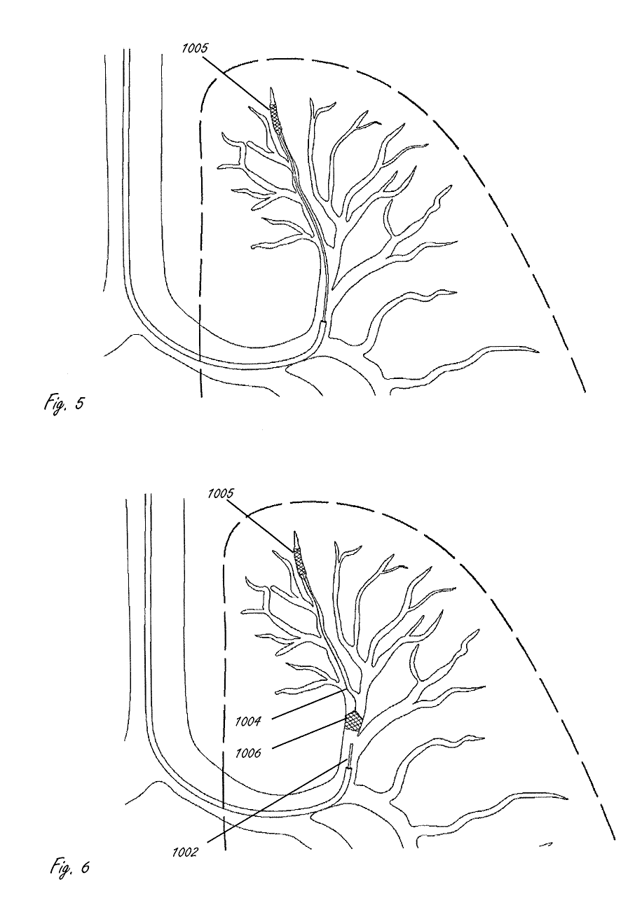

FIG. 4 illustrates an initial step in the delivery of a treatment device to a target location within the lung. Bronchoscope (1012) has been navigated and tracked into the airway leading to the emphysematous tissue to be treated. Once in place, delivery sheath (1002) is tracked distally into the emphysematous tissue. The delivery sheath should be advanced as far as practical, while avoiding potentially rupturing the parenchyma.

In some embodiments the distal end of the delivery sheath will comprise a tissue evaluation device which is used to identify emphysematous tissue. One such evaluation comprises the measurement of the electrical impedance of the tissue. Alternative means include but are not limited to, ultrasonic, and optical means. Electrode elements 1131 comprised on the distal end of the delivery sheath (1013) are used query the adjacent tissue as the device is delivered down the bronchi. If emphysematous tissue is observed, as would be the case in the illustration of FIG. 4, a distal anchor may be placed.

FIG. 5 illustrates a subsequent step (not necessarily immediate after) in the delivery of the device. As shown in FIG. 5, distal anchor (1005) has been deployed from the delivery sheath and has expanded into or towards its expanded configuration. Methods of deploying an expandable anchor from a delivery sheath are known, such as retracting a delivery sheath relative to an anchor whose position in maintained. The distal anchor optionally has a plurality of tines (i.e., sharp protrusions that puncture, hook into, or otherwise obtain traction) that engage the airway wall in which the anchor is deployed. In some embodiments there can be between 4-300 barbs or tines that engage the vessel wall, with larger numbers being preferred (but not required) because the load carried by the anchor will be better distributed as more tines are involved.

Distal anchor (1005) is configured to radially expand in response to expansion of the airway in which it is anchored. The anchor should be capable of 100%-700% of the maximum expansion expected of the airway in which it is deployed. Providing such expansibility will prevent the airway from expanding to a diameter that exceeds the ability of the anchor to remain engaged with the airway, resulting in a loss of anchoring.

A subsequent step (but not necessarily immediate after), as shown in FIG. 6, is to deploy the proximal anchor (1006) from the delivery sheath and expanding proximal anchor (1006). Tether (1004) can be seen extending between the distal anchor (1005) and the proximal anchor (1006). The delivery sheath (1002) can be withdrawn proximally to deploy the proximal anchor (1006). The tether (1004) maintains a mechanical connection between the distal anchor (1005) and the proximal anchor (1006).

FIG. 7 shows, after the proximal anchor has been deployed at a target location, a drive shaft (1003) can then be tracked through the bronchoscope or through the sheath contained within the bronchoscope so that its distal end engages with the interface (1008) of the socket (1007) in the proximal anchor (1006). In this embodiment, when the drive shaft engages with interface (1008), the drive shaft and the socket are rotationally coupled.

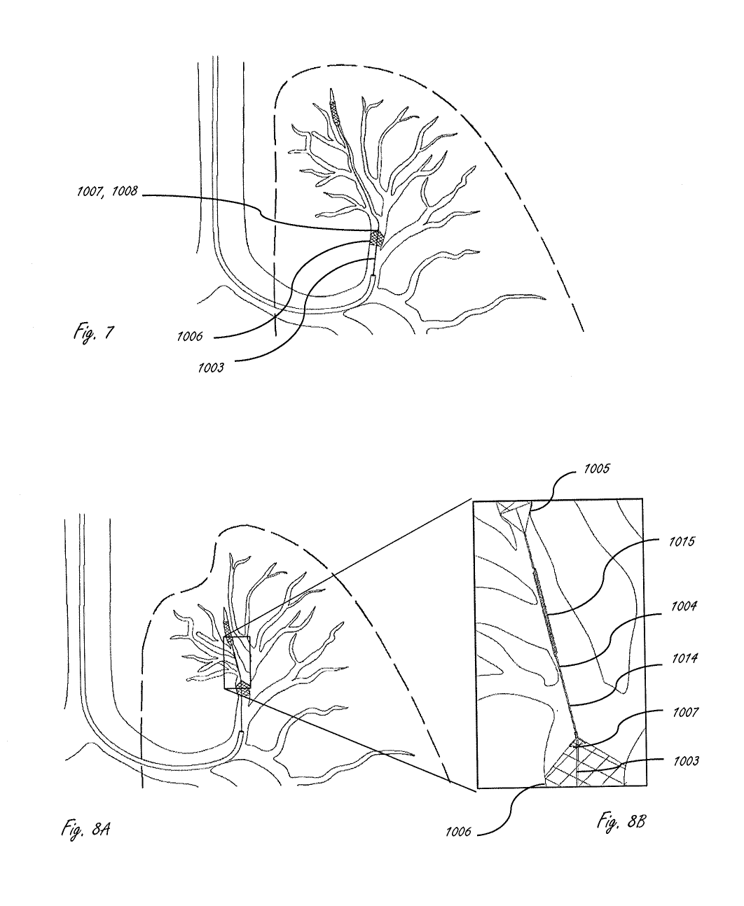

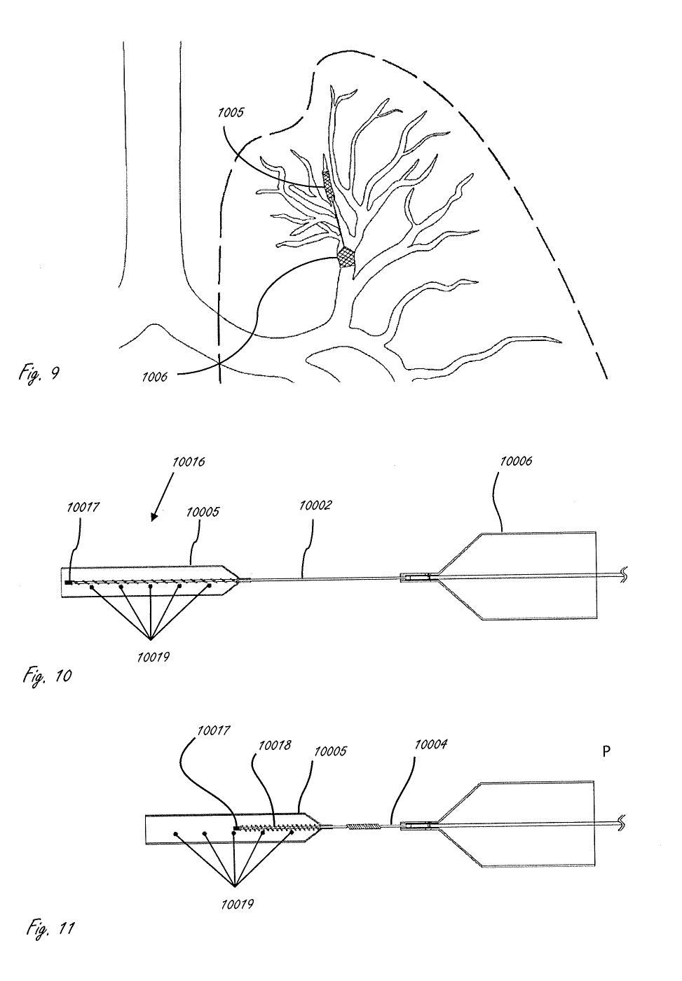

As shown in FIGS. 8A and 8B, the user then actuates (in this embodiment by rotating) the drive shaft (1003), causing the rotation to be transmitted through the socket (1007) and into the tether (1004). Rotating the drive shaft causes the tether to change configurations from a first configuration to a second configuration, which shortens the distance between the anchors. In this embodiment, as shown in the detailed view in FIG. 8B, the actuation causes a first portion (1015) of the tether to coil up into a non-straight configuration. This act of assuming a non-straight configuration causes the distal anchor (1005) to be drawn towards the proximal anchor into closer proximity to the proximal anchor (1006). The shortening of the distance between the distal anchor (1005) and the proximal anchor (1006) measured along the tether collapses the tissue between the anchor and has caused a volumetric reduction in the emphysematous portion of the lung.

FIG. 9 illustrates the treatment device in place within the lung after the bronchoscope has been removed. At this stage the final outcome of the lung volume reduction procedure can be observed.

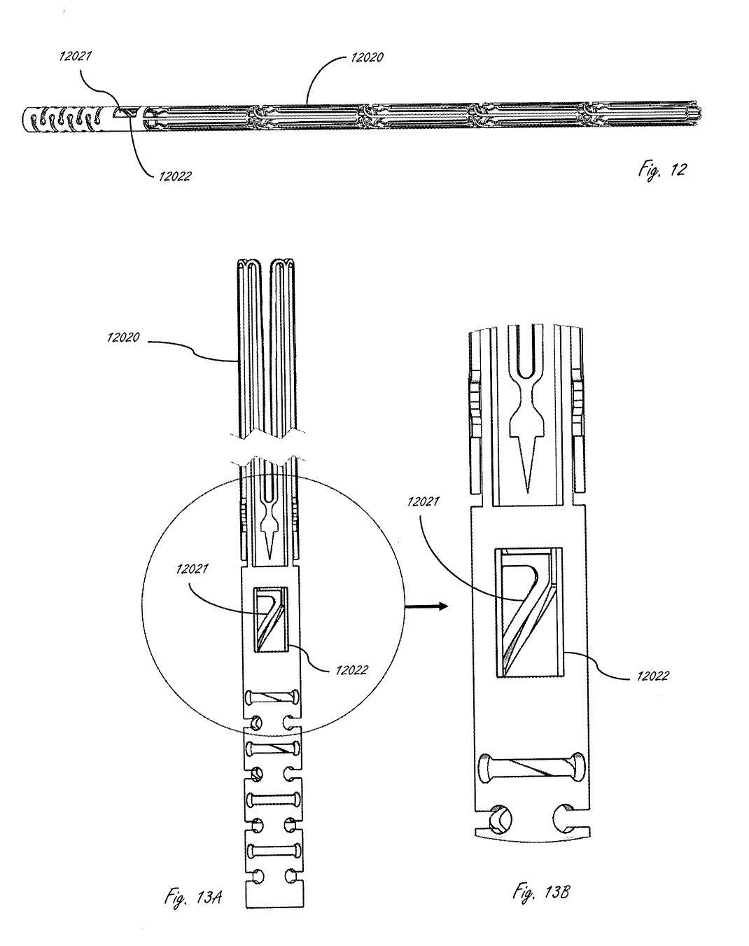

Some treatment devices herein include tension monitoring mechanisms. A tension monitoring mechanism is configured to allow the amount of tension that is applied to the tether to be monitored. FIGS. 10 and 11 illustrate a treatment device that includes a tension monitoring mechanism. Tension monitoring mechanism (10016) includes a central marker (10017) that is attached to the distal end of tether (10004), compression spring (10018) that is positioned between the central marker (10017) and a proximal region of distal anchor (10005), and a plurality of anchor markers (10019) that are fixedly attached to the distal anchor (10005) along the length of the distal anchor.

In FIG. 10, the lung volume reduction system is shown with no tension applied to tether (10004). As a result, the compression spring (10018) does not have a compressive load on it, and maintains a fully elongated condition. The distance between the anchors along the tether is a first distance. Referring to FIG. 11, the lung volume reduction system has been tensioned (using any of the tensioning mechanisms and methods herein) within the airways (not shown) in order to compress them and their associated parenchyma. The tension carried in the tether (10004) is transmitted to the compression spring (10018), which assumes a compressed condition as a result of this applied load. Because the spring constant k follows the typical linear relationship between applied load F and its degree of compression x, the spring equation F=k*x can be used to monitor the tension in the tether of the lung volume reduction system. A user may therefore monitor the position of the central marker (10017) relative to the position of the anchor markers (10019) in order to determine the tension on the lung volume reduction system. Additionally, the geometry of the compression spring (10018) can be controlled to the degree that the spring constant k can be known, allowing the tension in the lung volume reduction system to be known with accuracy. The central marker (10017) and anchor markers (10019) can be constructed from radiopaque materials so that they can be seen using fluoroscopy or other X-ray imaging techniques. Alternatively, they may be constructed to be visible via other methodologies (i.e. MRI, ultrasound, and the like).

The embodiment in FIGS. 10 and 11 also illustrates the manner in which the distal anchor is drawn closer to the proximal anchor when the tether is tensioned. The embodiment in FIGS. 10 and 11 is shown as manufactured, that is, outside of a lung. When the tether is tensioned, the tether changes configuration and becomes shorter as measured along the tether (as shown in FIG. 11). Because the tether is fixed to the distal anchor, the shortening of the tether pulls on distal anchor (10005) in the proximal direction “P” as shown in FIG. 11 (to the right in the figure). The axis of the tether stays the same when tensioned, even though the configuration may change. For example, the axis of the tether in FIGS. 10 and 11 is the same direction even though the configuration in FIG. 11 is wound up on itself. Tensioning the tether pulls the distal anchor in the proximal direction “P” towards the proximal anchor. In general, the distal anchor is pulled linearly towards the proximal anchor in the P direction. The distance between the distal anchor and proximal anchor measured along the tether is decreased, even though the actual length of the tether between the anchors remains the same (i.e., the tether winds up on itself, but the actual length of the tether between the anchors remains the same). Also, in the embodiment in FIGS. 10 and 11 (and some others herein), when the distal anchor is pulled towards the proximal anchor, its rotational orientation (out of a plane comprising the tether axis) relative to the proximal anchor stays the same as it is pulled towards the proximal anchor. For purposes of simplification, when the embodiments herein are described as being configured such that the distance between distal and proximal anchors can be reduced or increased, they are being described in their as-manufactured configurations (e.g., the embodiment shown in FIGS. 10 and 11), or what happens when actuated on a tabletop outside of a lung.

In contrast to the descriptions in the paragraph above, a device that has an initial straight configuration and is configured to bend with a pullwire or other means, for example, does not have a distal anchor that moves towards a proximal end or proximal anchor as described herein. For example, when bent, the tether (bent) axis is not in the same direction as when the device is straight. Additionally, the distal end of the bend device does not have the same rotational orientation relative to the proximal end. These are examples of structural differences between devices herein and devices configured to bend when actuated. Again, the structural descriptions and how the devices are configured reflect the devices when they are outside of the human body, in their as-manufactured configuration (although the devices are intended to change configurations in the same or similar manner when in use within the lung). When in use, the shortening of the distance between the distal anchor (10005) and the proximal anchor (10006) measured along the tether causes a volumetric reduction in the emphysematous portion of the lung.

In some alternative embodiment to that shown in FIGS. 10 and 11, the distance central marker (10017) travels can also be used to determine how much the distance between the anchors changes, if a correlation exists between the distance central marker (10017) travels and the distance the tether shortens in the portion that changes configuration.

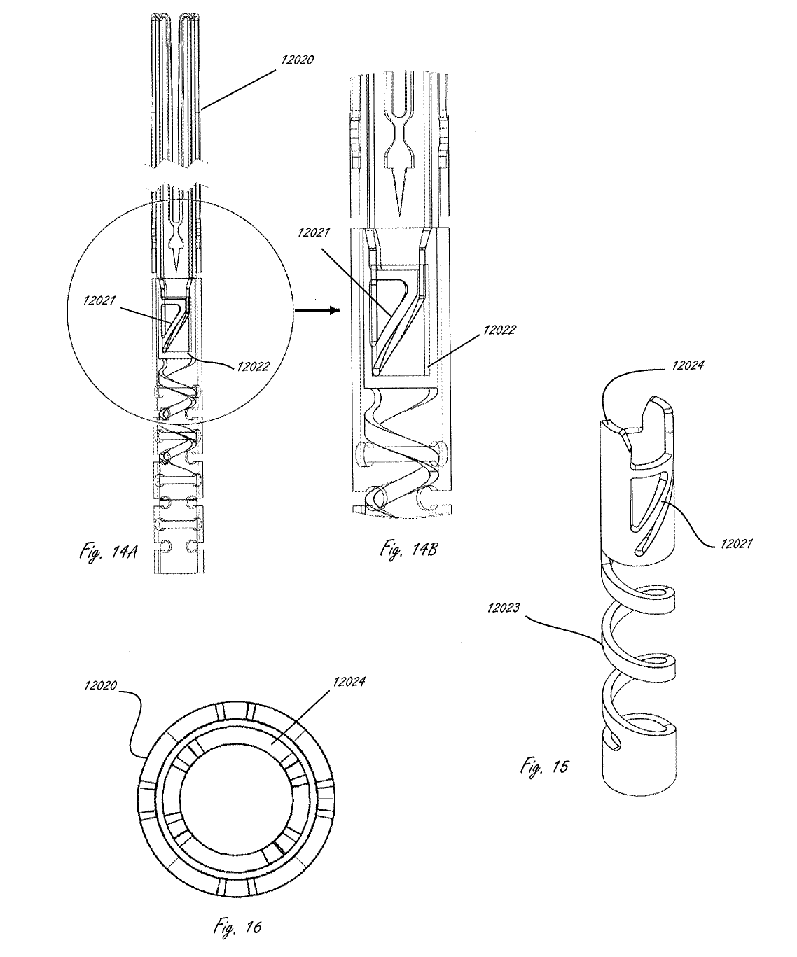

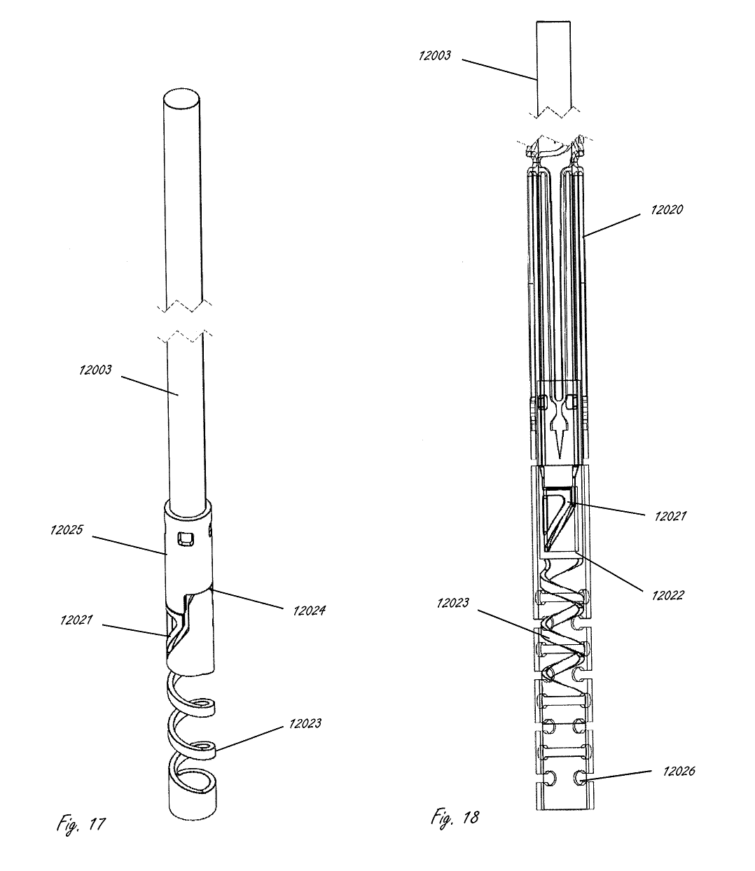

FIGS. 12 through 18 illustrate an exemplary mechanism configured to hold and adjust tension on a tether. This design includes a stent like tube (12020) shown in FIG. 14A, into which an expandable structure is cut. In one end of this tube a window (12022) is cut as illustrated in FIG. 14A and corresponding inset FIG. 14B. The spring like element of FIG. 15 (12023) is cut into a smaller tube that fits within the distal end of the larger tube 12020 and rests upon the flange of FIG. 18 (12026) fixed within the inner diameter of the outer tube. This flange prevents the element from moving beyond the tube under tension but allows for the element to rotate. A tab (12021) is cut into the smaller tube, which is then shape-set to extend slightly out of the surface of the inner tube and into that of the outer tube. The tab fits within the window of the outer tube. When the drive shaft of FIG. 17 (12003) with interface tip (12025) is advanced to the interface of the spring element (12024) and is rotated the element twists and the tab allows for rotational motion in only one direction. The orientation of the tab results in any rotational motion that is opposite of that which is desired being halted. This feature allows for tension to be increased or decreased and held in place. By rotating the tether it twists and foreshortens drawing the distal anchor it is affixed to in towards this proximal ratcheting structure. Should it be desired to release the tension in the line the drive shaft can be advanced further as to depress the spring of the spring element within the outer tube. When this spring is depressed the raised tab is forced to lay flat as it is disengages from the outer tube window it extends into. As it is disengaged it is free to spin freely within the stationary outer tube. This design allows for a completely adjustable and reversible tension to be applied to tethers within the airways of the lung.

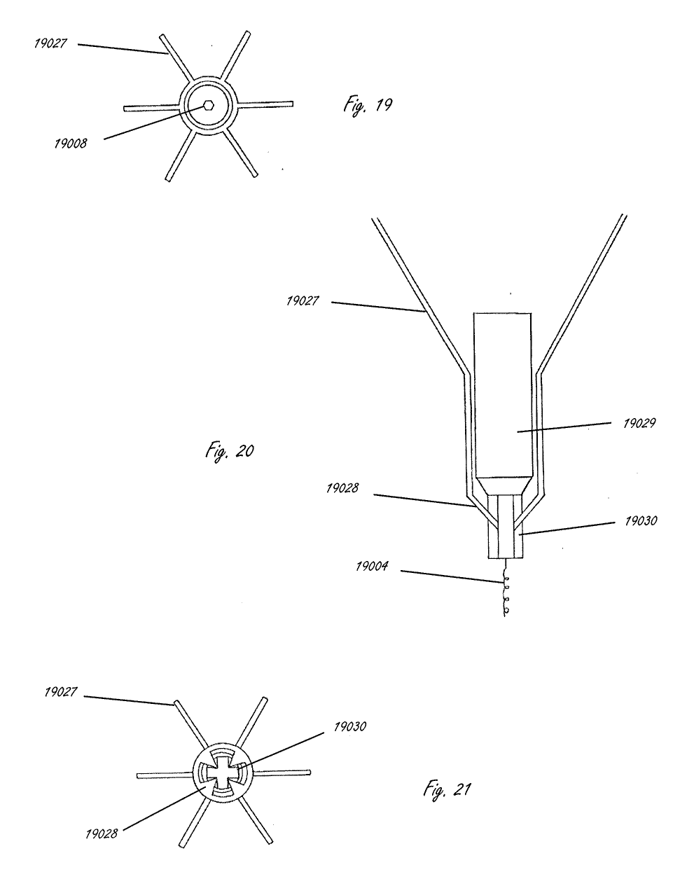

FIGS. 19-21 illustrate an exemplary embodiment of an apparatus configured to tighten the tether and thus reduce the volume of the lung. Only a portion of the treatment device is shown in this embodiment for clarity. FIG. 19 shows a back view, FIG. 20 shows the side view, and FIG. 21 shows the front view. In the side view of FIG. 20, the distal direction is downward. The treatment device includes a proximal anchor, which includes an expandable stent like structure (19027) coupled to an internal ratcheting shaft (19029). The ratcheting shaft (19029) has an interface (19008) that is configured to receive a drive shaft therein such that the drive shaft and ratcheting shaft (19029) are rotationally coupled. As the shaft rotates within the outer tube the keyed channels machined into the shaft (19030) also rotate. The outer tube is shaped in such a way that there are tabs (19028) set down to interface with the keyed channels of the inner shaft. The outer stent like structure remains rotationally fixed within the tissue and as an external torque is applied rotating the shaft the tabs snap into the keyed channels of the ratcheting shaft. These tabs are designed in such a way to provide just enough resistance to ensure no rotation in either direction without the introduction of external force. This allows for the operator to selectively set the tension within the tether attached to the distal portion of the inner shaft. As the shaft is rotated, the motion is transferred to the tether (19004). By setting the external stent like structure as well as the distal anchor in place, the torque applied to the tether is captured within the line resulting in a reduction of the tether length, thus reducing the volume of the lung. The drive shaft can then at any time be repositioned within the interface to modify the tether configuration to increase or decrease the distance between the anchors.

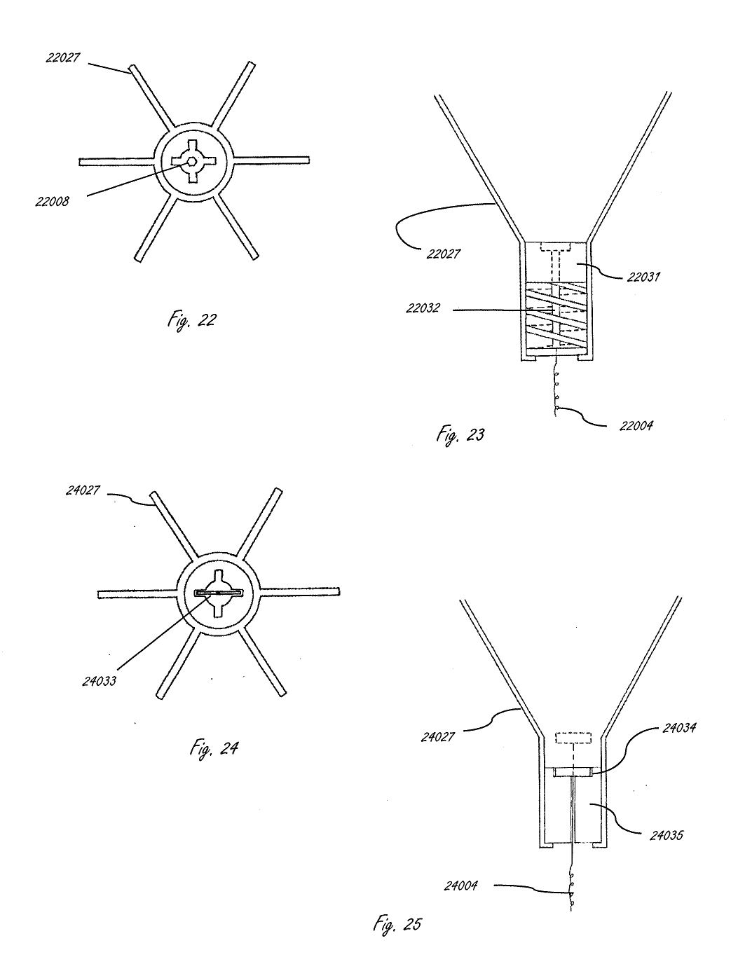

FIGS. 22 and 23 illustrate another exemplary embodiment of an apparatus configured to tighten the tether and thus reduce the volume of the lung. FIG. 22 is a top view and FIG. 23 is a side view. Only a portion of the treatment device is shown. In this embodiment there is an outer tube with an internal spring loaded collar (22031) resting on a flange at the distal tip of the tube. The tether (22024) is affixed to the distal end of a core shaft (22032) within the spring element. A drive shaft is inserted into the interface (22008) with an integrated feature that depresses the collar releasing the catch feature of the inner shaft from the pocket it fits into within the collar. By releasing this feature the shaft is free to rotate in both directions. The drive shaft is used to apply torque to the tether through this inner shaft. Once the desired torque is reached the drive shaft is withdrawn which allows the spring loaded collar to again snap over the catch feature of the inner shaft preventing any additional rotation or release of tension. In this way the tether is maintained in a desired configuration. The drive shaft can then at any time be repositioned within interface 22008 to modify the tether configuration to increase or decrease the distance between the anchors.

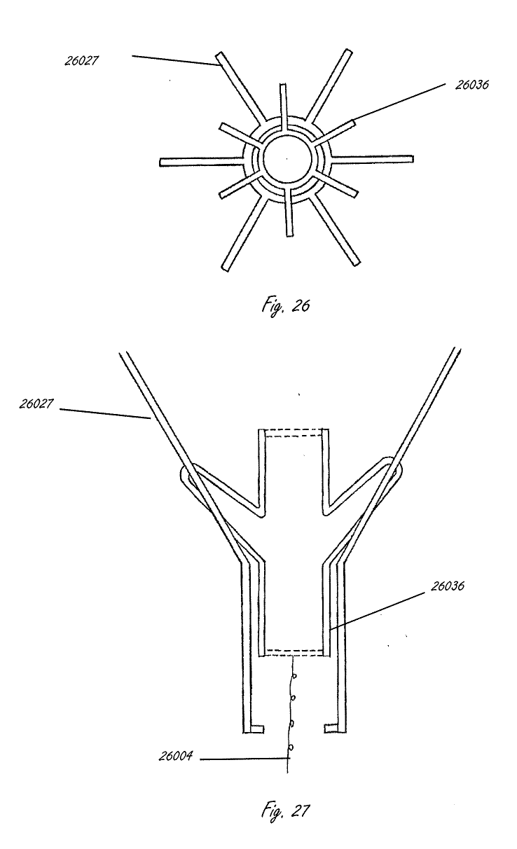

FIGS. 24 and 25 illustrate another embodiment of an apparatus configured to tighten the tether and thus reduce the volume of the lung. FIG. 24 shows a top view and FIG. 25 shows a side view, and only a portion of the treatment device is shown for clarity. In this embodiment the proximal anchor includes an outer stent like structure (24027) cut into a tube and a pocketed collar (24033) that rests within the inner diameter of this tube. The tether (24004) runs through the inner channel of the collar (24035) that is fixed within the outer tube. This tether terminates in a catch (24034) that is shaped in such a way that it fits within the pocket of the collar (24033). A drive shaft is advanced to the collar and interfaces with the catch which is pulled proximally slightly to free it from the pocket it rests within. Torque is then applied to the catch which is transferred to the tether to foreshorten it. Once the desired tension is reached the catch is placed back into the pocket of the collar. The tension within the line keeps the catch seated within the collar which in turn prevents any additional rotation or release of tension within the tether. In this way the tether is maintained in a desired configuration. The drive shaft can then at any time be repositioned within the interface to modify the tether configuration to increase or decrease the distance between the anchors.

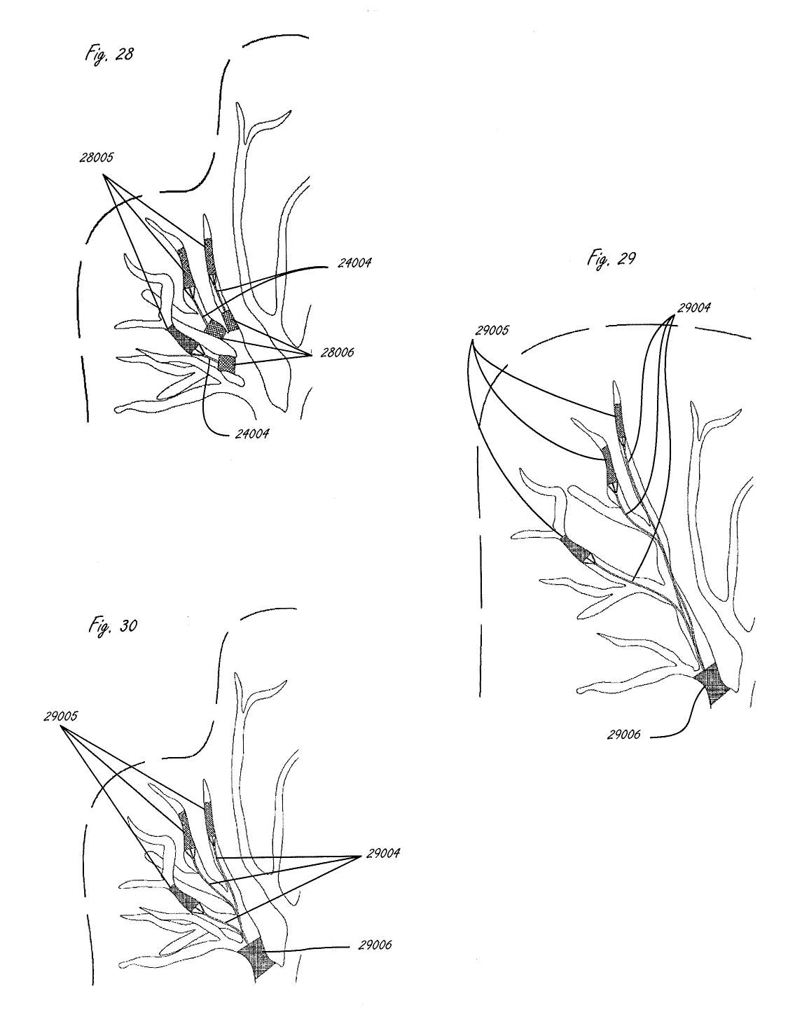

FIGS. 26 and 27 illustrate another embodiment of an apparatus configured to tighten the tether and thus reduce the volume of the lung. FIG. 26 shows a top view and FIG. 27 shows a side view. Only a portion of the treatment device is shown for clarity. This embodiment includes two stent like tubular elements. The outer tubular element (26027) is set and an internal tubular element (26036) is inverted in such a way that the expanded features of this inner tubular element interfaces with those of the outer tubular element. This interface prevents any rotation until a drive shaft is used to introduce the necessary force to rotate the inner tubular element. The internal stent rotates within the stationary external tubular element and the inverted members snap out in between each longitudinal element of the external tubular element. Once the desired torque is reached the drive shaft is withdrawn and the interference between the elements of the two tubular elements prevents any additional rotation or loss of tension in the tether (26004). In this way the tether is maintained in a desired configuration. The drive shaft can then at any time be repositioned within the interface to modify the tether configuration to increase or decrease the distance between the anchors. Alternatively, the drive shaft may be configured to reversibly lock to the internal tubular element. In such an embodiment the internal tubular element is drawn proximally to rotate, either for foreshortening or lengthening. Upon completion of the adjustment step the drive shaft is released and the tension between the distal and proximal anchors holds the inner tubular element in the rotationally interlocked position relative to the outer tubular element.

FIG. 28 illustrates an embodiment of a method of reducing the volume of a lung by positioning a plurality of separated treatment devices with the lung. In this embodiment each of the individual treatment devices includes a distal anchor (28005), a proximal anchor (28006), and a tether (28004), similar to the embodiment shown in FIGS. 1A-1C and FIG. 2. Each of the individual treatment devices can be actuated with a drive shaft to control the tension in the respective tether and thus the distance between the respective distal and proximal anchors. The physician may evaluate the resulting tissue response and may decide to continue treatment by increasing, decreasing, or maintaining the tension on each tether. The tension may be applied to all tethers uniformly, or may be applied individually depending on the adjustable proximal anchor design. Furthermore, the physician may choose to completely eliminate tension on the tether between anchors if it is no longer desired.

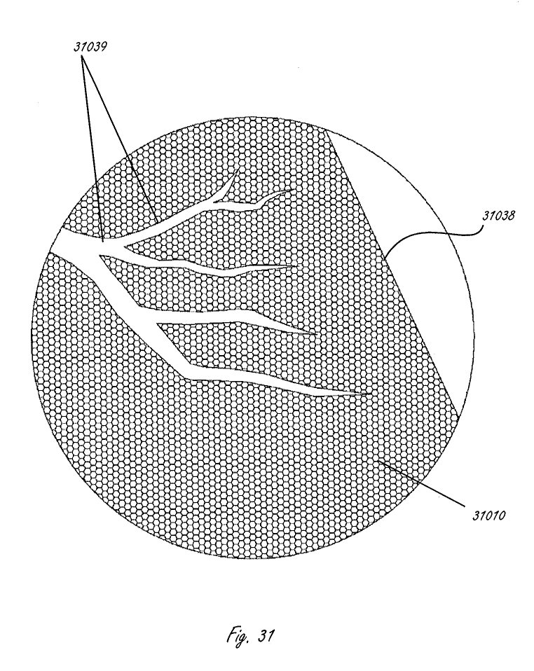

In some embodiments a treatment device includes a plurality of distal anchors coupled to one proximal anchor. A tensioning component secured to the proximal anchor is actuated to modify the tension in the plurality of tethers. Each of the plurality of tethers can be individually tensioned or they can be tensioned together. The configuration of each of the tethers can thus be different, or the tethers can all change configurations to the same extent. FIG. 29 illustrates an exemplary embodiment in which the treatment device has been positioned within the lung and the plurality of distal anchors and the single proximal anchor are expanded and anchored to respective lumens. FIG. 30 illustrates the treatment device after each of the tethers has been tensioned, which has pulled each of the distal anchors towards the proximal anchor. In this exemplary embodiment each tether is coupled to the proximal anchor at substantially the same location. In this embodiment of an adjustable anchor system for lung volume reduction, the apparatus includes a plurality of distal anchors (29005), an adjustable proximal anchor (29006), and tethers (29004) connecting the distal anchors and the adjustable proximal anchors. As previously discussed, the tethers may also be tightened in a stepwise fashion over time to provide maximal lung volume reduction while minimizing the chance of tearing of the parenchyma and other unwanted side effects (i.e. inflammation, bleeding etc.). FIG. 30 shows the apparatus after the tethers (29004) have been tightened and the delivery device removed. Because all of the anchors (29005) are tethered to a single adjustable proximal anchor (29006), they will all be drawn together towards a single location. The physician may evaluate the resulting tissue response and may decide to continue treatment by increasing, decreasing, or maintaining the tension on each tether. The tension may be applied to all tethers uniformly, or may be applied individually depending on the adjustable proximal anchor design. Furthermore, the physician may choose to completely eliminate tension on the tether between anchors if it is no longer desired.

The method shown in FIG. 28 may have an advantage of use when the lung tissues are more diseased and are not able to support the localized loading associated with a single location adjustable proximal anchor, such as in the embodiment shown in FIGS. 29 and 30. The method shown in FIG. 28 also allows the physician to only need to consider a single airway when placing each of the devices. Likewise, tensioning could be a simpler procedure because only one tensioning line is present in the airway, whereas the design shown in FIGS. 29 and 30 could require the user to discriminate between tensioning mechanisms for each tether. Alternatively, in some lungs there may not be enough healthy lumens in which to anchor more than one proximal anchor. In those situations it may not be safe to use more than one anchoring device, each with its own proximal anchor. In these situations a single proximal anchor design may provide the benefit of being able to be anchored in a single healthy tissue lumen while still being connected to a plurality of distal anchors. For example, in the embodiment in FIGS. 29 and 30, only proximal anchor (29006) need be anchored in healthy tissue. In FIG. 28, proximal anchor (28006) is anchored in healthy tissue. But if in FIG. 28 three healthy lumens cannot be detected, a choice of the procedure may be to use a single proximal anchor device.

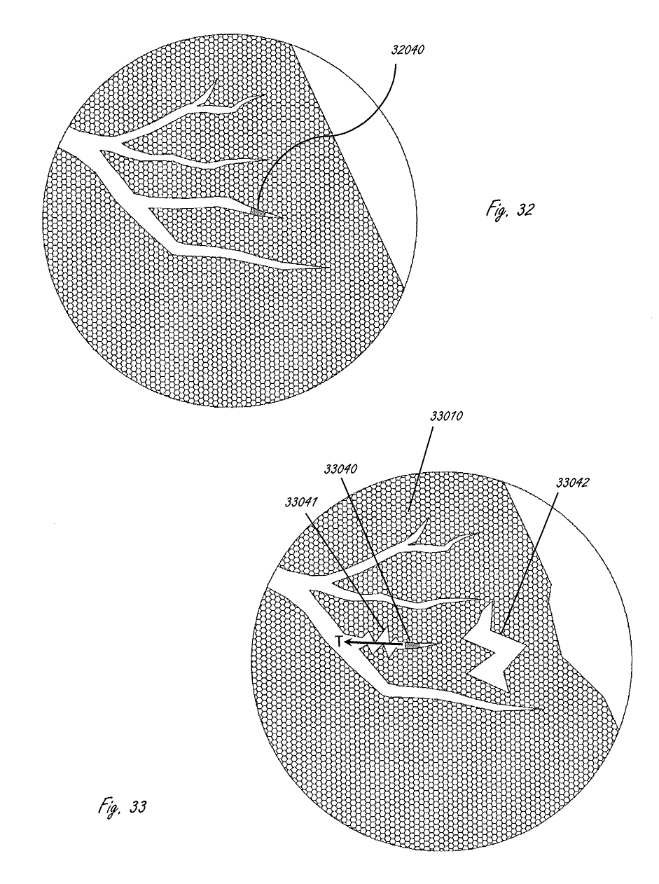

FIGS. 31-36 illustrate methods of use that can be used when placing a plurality of distal anchors in different lumens, regardless if one or more proximal anchors are used. Proximal anchors are thus not shown for clarity, but may be a single proximal anchor or a plurality of proximal anchors as described herein. FIG. 31 illustrates a sectional view of a portion of an emphysematous lung. FIG. 31 shows a surface of the lung, or visceral pleura (31038), a network of airways (31039), and a finer structure of bronchioles, blood vessels, and alveolar tissue herein referred to as the parenchyma (31010). FIG. 32 illustrates a single lung distal anchor (32040) configured for lung volume reduction. Tension (T) is applied to the anchor in FIG. 33 via a proximal anchor and tether (not shown for clarity), causing the adjacent airway (33041) to foreshorten. The tension is transmitted to the parenchyma (33010) surrounding the airway. The parenchyma (33010) is a delicate tissue, and in this case, the tension has exceeded the tensile strength of the parenchyma (33010), resulting in a tear (33042). The tear (33042) causes a degree of mechanical isolation between the airway containing the anchor and the outer extremities of the adjacent parenchyma, preventing the applied tension from reaching those extremities. As a result, the lung volume reduction is smaller than if no tear had occurred. Tearing is an undesired consequence and should be prevented.

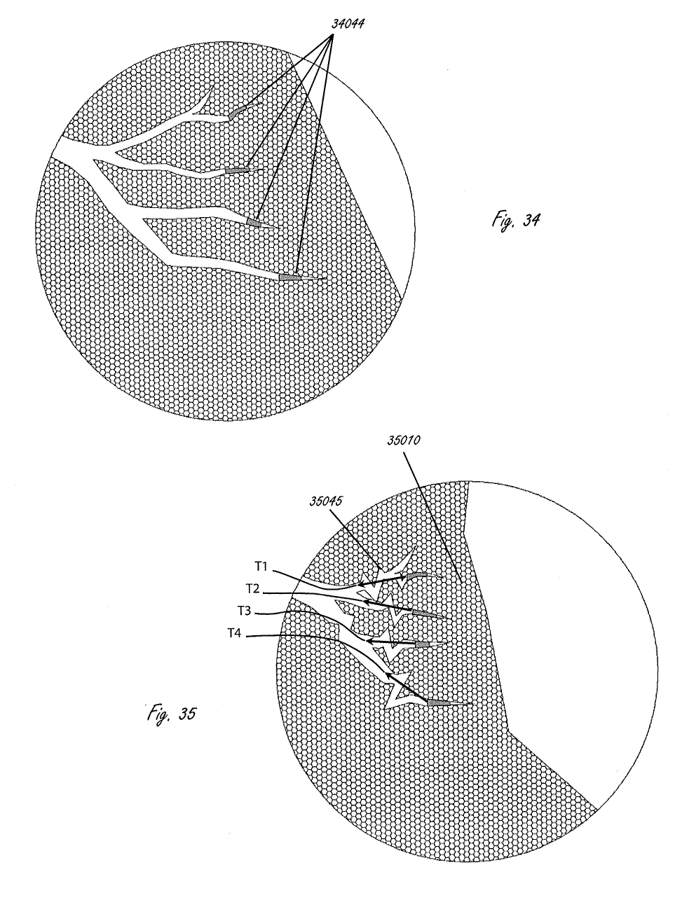

FIGS. 34 and 35 illustrate an exemplary embodiment of a method using a plurality of distal anchors for lung volume reduction. In this embodiment a plurality of lung anchors (34044) are utilized. Tensions (T1, T2, T3, T4) are applied by tethers (see FIG. 35) interfacing either a single or multiple proximal anchors (not shown) causing the adjacent airways (35045) to foreshorten. The tensions are transmitted to the parenchyma (35010) area surrounding the airways (35045). While the parenchyma tissue (35010) remains delicate, the applied loads are spread over a larger area, and do not exceed the tensile strength of the parenchyma. As a result, no tear is formed, and the applied tensions can reach the outer extremities of the parenchyma. A much more effective lung volume reduction is achieved by avoiding tearing of the parenchymal tissue.

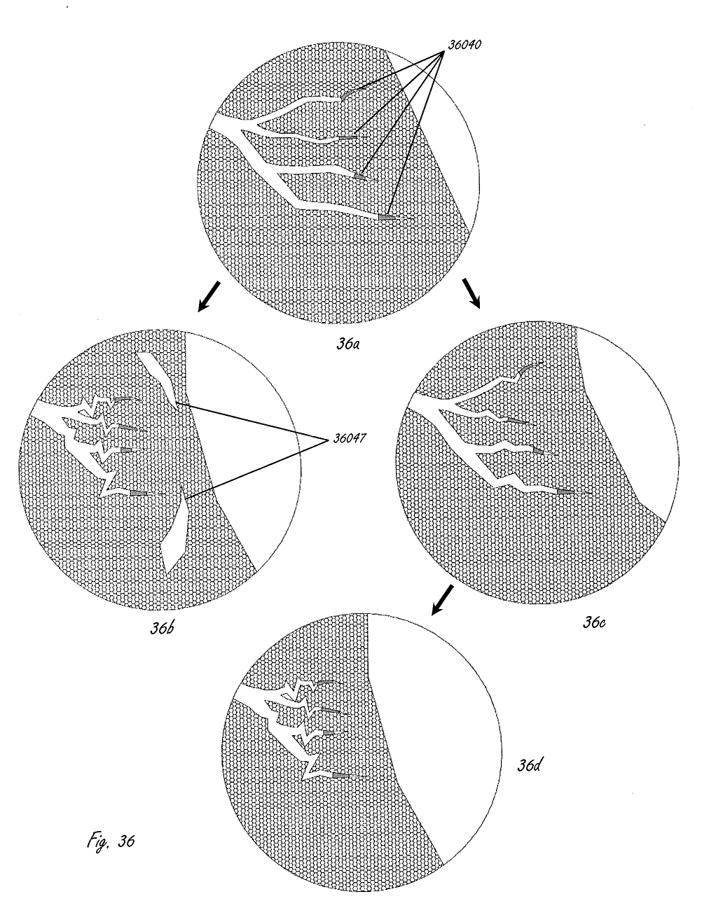

FIGS. 36A-36D illustrate an embodiment in which tension in respective tethers can be individually controlled. This embodiment also illustrates advantages of timing aspects of tensioning a plurality of tethers. FIG. 36A illustrates a portion of an emphysematous portion of the lung, wherein a plurality of lung anchors (36040) have been placed. FIG. 36B shows a potential result if a high level of tension is immediately placed on the anchors (36040). Tears (36047) are formed due to the high level of tension applied resulting in a reduced ability to reduce lung volume as similarly discussed for FIG. 33. Alternatively, FIGS. 36C and 36D illustrate a result if the tension to the tethers and anchors is applied stepwise and sequentially. An initial tension applied to all anchors as shown in FIG. 36C is significantly less than what will cause tearing in the parenchyma. After the initial tensioning, a period of time is allowed to elapse before applying additional tension. After the period of time has elapsed, additional tensioning is applied to all of the anchors, as shown in FIG. 36D. By performing the tensioning in a stepwise and sequential fashion, healing can occur in the tissue between tensioning events, which will allow greater ultimate deformation in the tissue without tearing. Another advantage of this stepwise tensioning is that any inflammation or other biological response from each tensioning event can subside before performing the next tensioning event. Additionally, imaging studies (e.g., X-ray, CT, MRI, and the like) may be performed between tensioning events to evaluate the impact of the previous tensioning event, and provide guidance for further tensioning events. Varying levels of tension may be applied to each anchor in order to maximize its reduction in lung volume, while preventing tearing of the parenchyma. In some situations it will be appropriate to perform the procedure in either a stepwise or a sequential fashion. In some embodiments in which stepwise and sequential tensioning are performed, one proximal anchor is used, and in some embodiments a plurality of proximal anchors are used.

In some of the embodiments herein, a tensioning controller is used to modify the tension in a plurality of tethers. FIGS. 37-44 illustrate exemplary embodiments in which a plurality of tethers can be controlled with a single tensioning controller. FIG. 37 is a top view and FIG. 38 is a side view of one embodiment of a tensioning apparatus configured to tighten a plurality of tethers of a treatment device to draw the distal anchors closer to a proximal anchor and reduce the volume of the lung. The tensioning apparatus is also configured so that the tension in the tethers can be modified repeatedly over time even after a delivery device has been removed from the lung. In this embodiment a drive shaft can be inserted into interface (37048) and motion clockwise or counter-clockwise is translated into radial motion of the main geared collar (37049) shown in the side view of FIG. 38. The teeth of this collar (37050) interface with the gear (37051) of each tether spool (37052). As the drive shaft and collar are rotated, the tether (37004) is either wound or unwound for each spool depending on the direction of rotation. Winding or unwinding the tether decreases or increases the distance between the anchors as described herein.

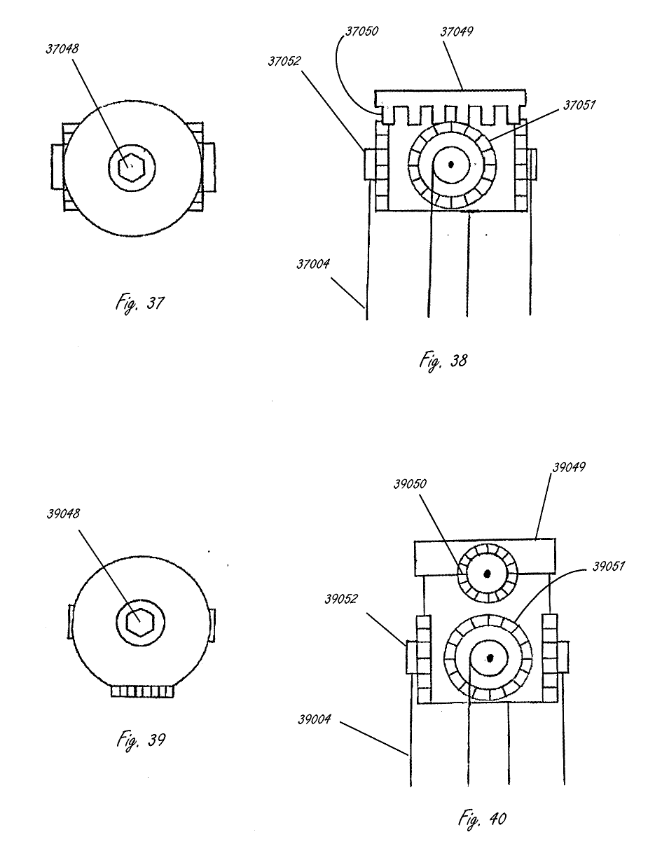

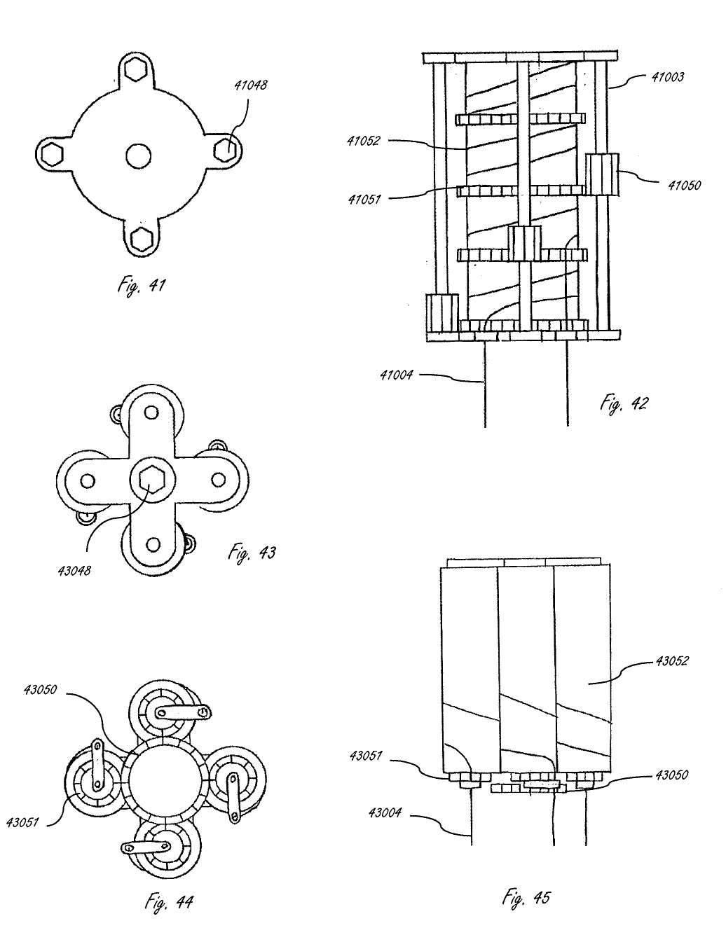

FIGS. 39 and 40 illustrate an alternative embodiment of a tensioning controller configured to modify the tension in a plurality of tethers. In this embodiment the tensioning controller is configured to be able to individually tension each tether, rather than tensioning all tethers at the same time. FIG. 39 shows a top view and FIG. 40 shows the side view. This embodiment in similar to the embodiment in FIGS. 37 and 38 but in this embodiment the spool engagement is different. In this design the teeth of the collar (39050) are on a gear mounted on the collar. The collar (39049) is configured to be rotated until it is over the particular spool (39052) for which tension is desired. The collar is then depressed which engages that particular spool gear (39051). The collar gear is connected to the drive shaft interface (39048) via a worm drive. When the collar is depressed and the drive shaft rotates, so does the collar gear. This motion is transferred to the spool which winds or unwinds the particular tether (39004) depending on the direction of rotation. Each tether can thus be individually tensioned. This allows each of a plurality of distal anchors to be individually controlled. In alternative embodiments the tensioning controlled is configured to tension more than one tether at the same time, but is also configured to not tension one or more other tethers at that time.

FIGS. 41 and 42 illustrate an exemplary embodiment of a tensioning controller configured to tension tethers individually. FIG. 41 shows a top view and FIG. 42 shows a side view. In this embodiment a drive shaft (41003) is inserted into a drive shaft interface (41048) thereby connecting that interface to a spool drive (41050). As each shaft is rotated its corresponding spool (41052) also rotates as it is interfaced to the spool drive through a gear (41051) which winds and unwinds the tether (41004). While this could be adapted to any number of spools, this embodiment illustrates a configuration of 4 different spools that can be utilized individually to tension individual tethers.

FIGS. 43-45 illustrate an alternative embodiment of a tensioning controller configured to tension tethers together. FIG. 43 is a top view, FIG. 44 is a side view, and FIG. 45 is a bottom view. In this design a core rod (not shown) connects the drive shaft interface (43048) to the collar gear (43050). All of the spools (43052) can be advanced on its center shaft which engages its spool gear (43051) with the collar gear. As the gear is rotated, whichever spool is engaged also rotates winding or unwinding the tether (43004) to draw tension in the lines.

FIGS. 46-49 illustrate two tether and anchoring apparatuses. FIGS. 46 and 47 are side views, respectively, of one embodiment. In this embodiment, in use, a cleating system (46053) is placed over a bifurcation of an airway within the lungs. The barbed catch features (46055) (see FIG. 47) interface with the tissue at the bifurcation to help prevent the cleat from backing off or coming loose from the tissue once positioned. The tether (46004) is affixed to a distal anchor within the airway and the proximal end of the tether is fed through the interface with the cleat (46054). This interface is configured in such a way that it ratchets over the ball on a line design of the tether. As tension is pulled on the tether the balls advance through the cleat, which snaps over each capturing the line and ensuring the tension within the line is maintained.

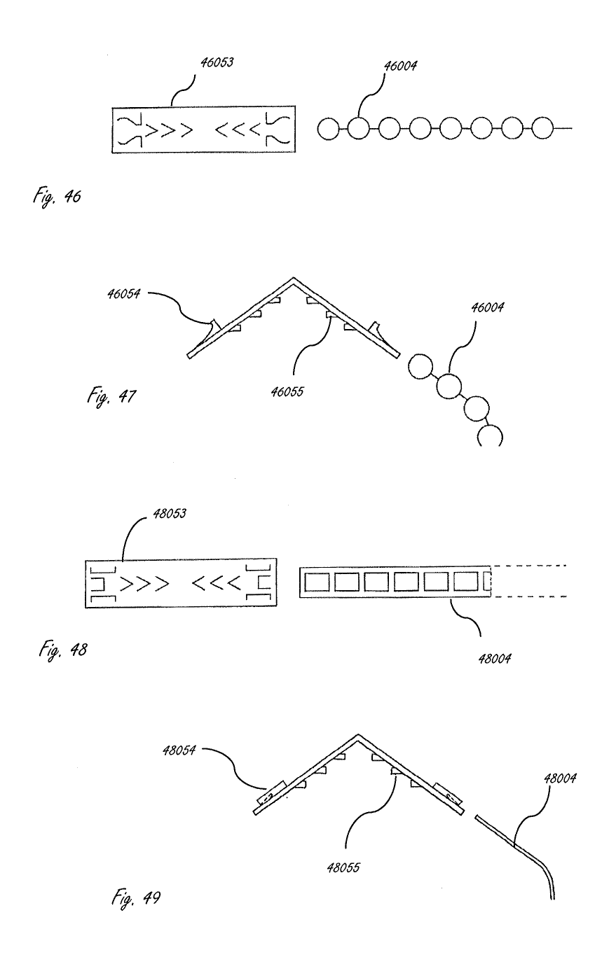

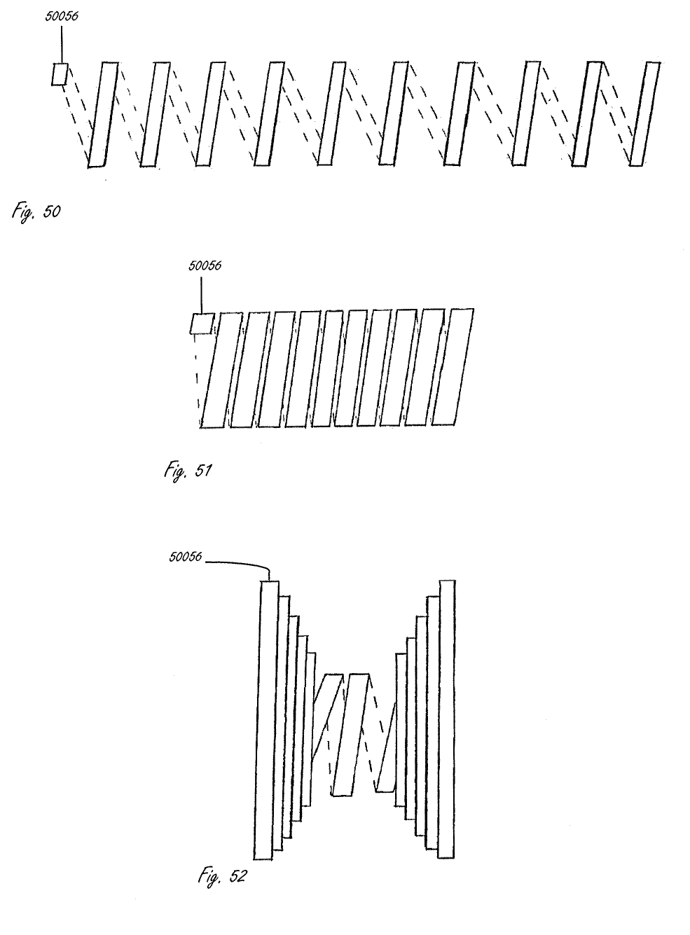

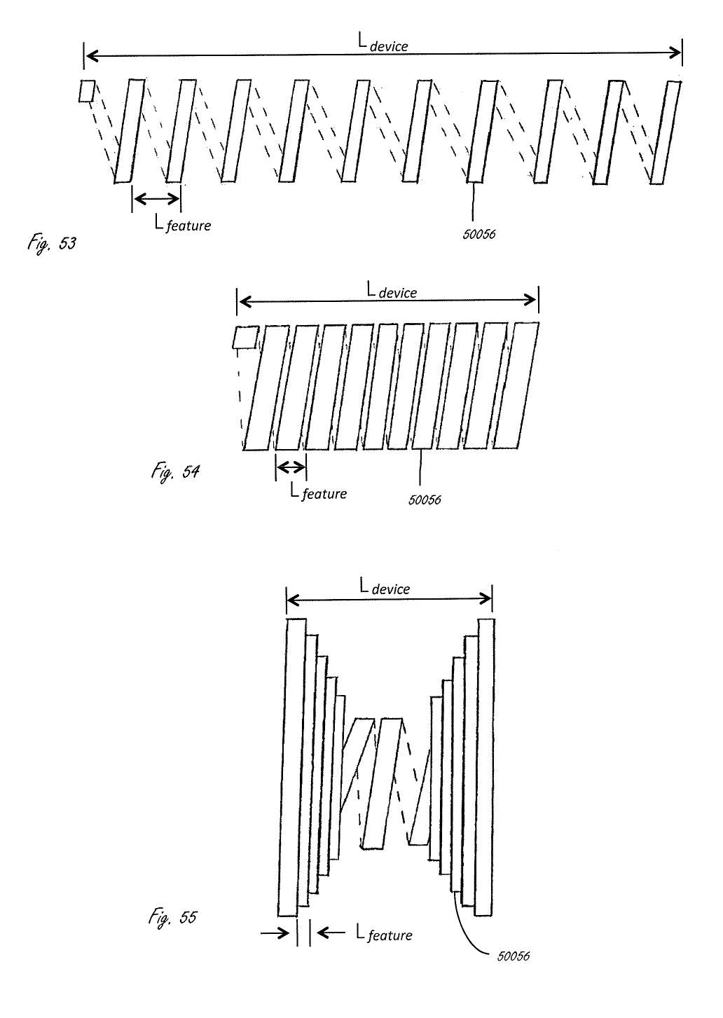

FIGS. 48 and 49 illustrate an alternative embodiment of a tether and anchoring apparatus, with top and side views, respectively. This design is similar to that illustrated in FIGS. 46 and 47 with the exception of the tether design and its interface with the cleat. As opposed to the balls on a line design this embodiment utilizes a ladder like design (48004) and the interface with the cleat (48053) has a step that is extended. As the tether is advanced into the cleat a raised stop (48055) snaps into each rung of the ladder holding the line in place and preventing the tension within the line from being released.

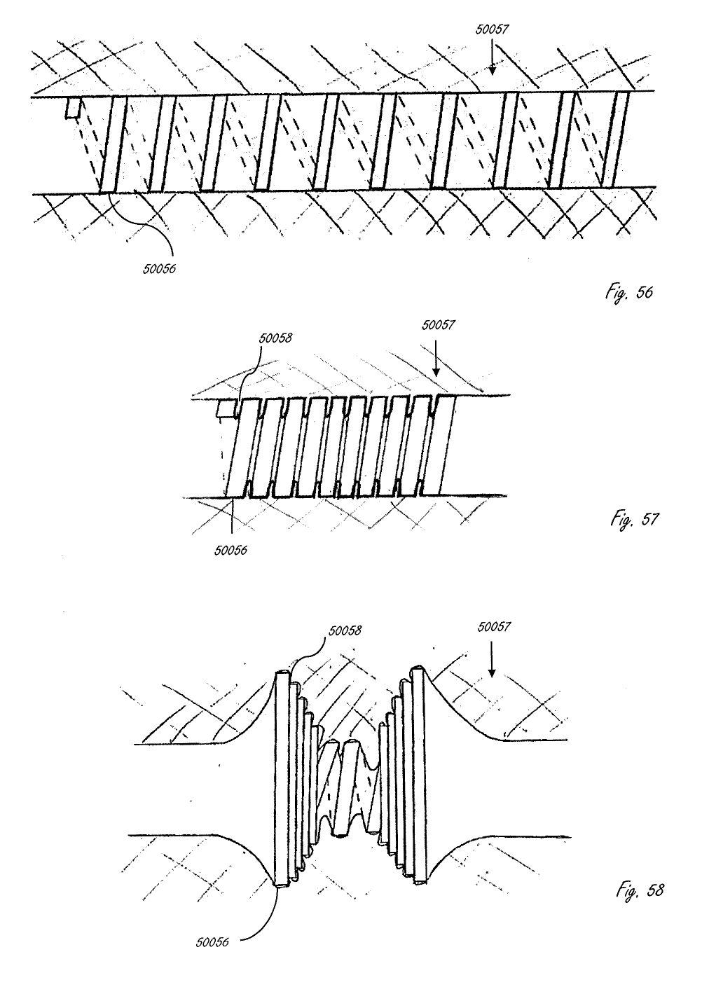

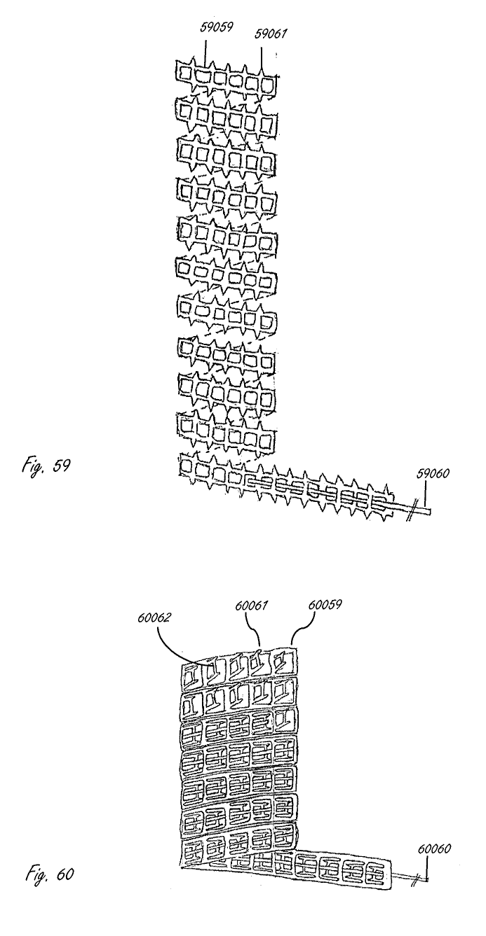

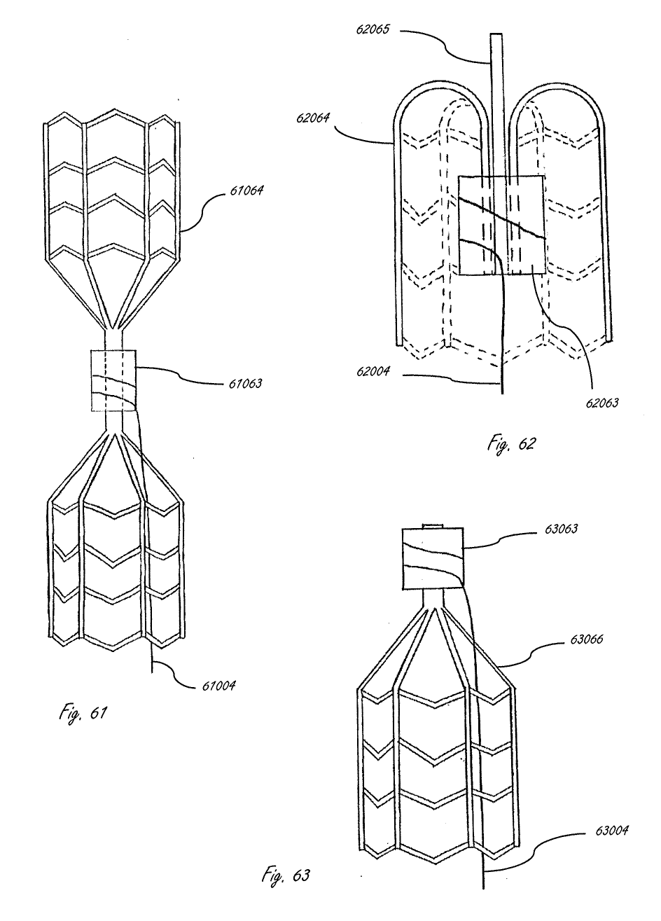

In some embodiments the tether comprises a spring or spring-like element (generally referred to herein as a “spring”). The spring can be stretched to an extended length, as shown in the exemplary embodiment in FIG. 50, and released. This device could be shape-set to have a relaxed state resembling that of a helix, such as is as shown in FIG. 51, where the edge of each element (50056) comes into contact with itself along the trailing edge as is wraps around the spiral path. The device could also be set to resemble a torsional spring, such as in the exemplary embodiment shown in FIG. 52. In this torsional spring configuration the longitudinal elements on opposing ends of the device lay over one another as they wrap in ever increasing diameters. While the helical and torsional spring designs could be stretched prior to delivery and appear to have the same pattern, when the two designs relax the amount they foreshorten as well as radial expand will vary. FIG. 53 illustrates how a device with longitudinal element wrapping in a spiral axis and stretched would appear. The overall device length (L_{device}) and feature length (L_{feature}) will be greater than the relaxed stated. As the device is released there is uniform engagement along the length of the device as the total length of the device and the feature length reduce. FIG. 54 shows this foreshortening for the helical design, while FIG. 55 illustrates the foreshortening of a torsional spring configuration. As the elements draw together from the spring force stored within the device, the tissue along the airway wall surrounding the device (50057), seen in FIG. 56, is engaged and drawn together. This uniform engagement of the tissue (50058) occurs between the trailing edges of the elements as shown in the reconfigured states shown in FIGS. 57 and 58. As the tissue is engaged and the feature length simultaneously foreshortens, the length of the tissue of the wall also reduces, reducing the volume of the lung.

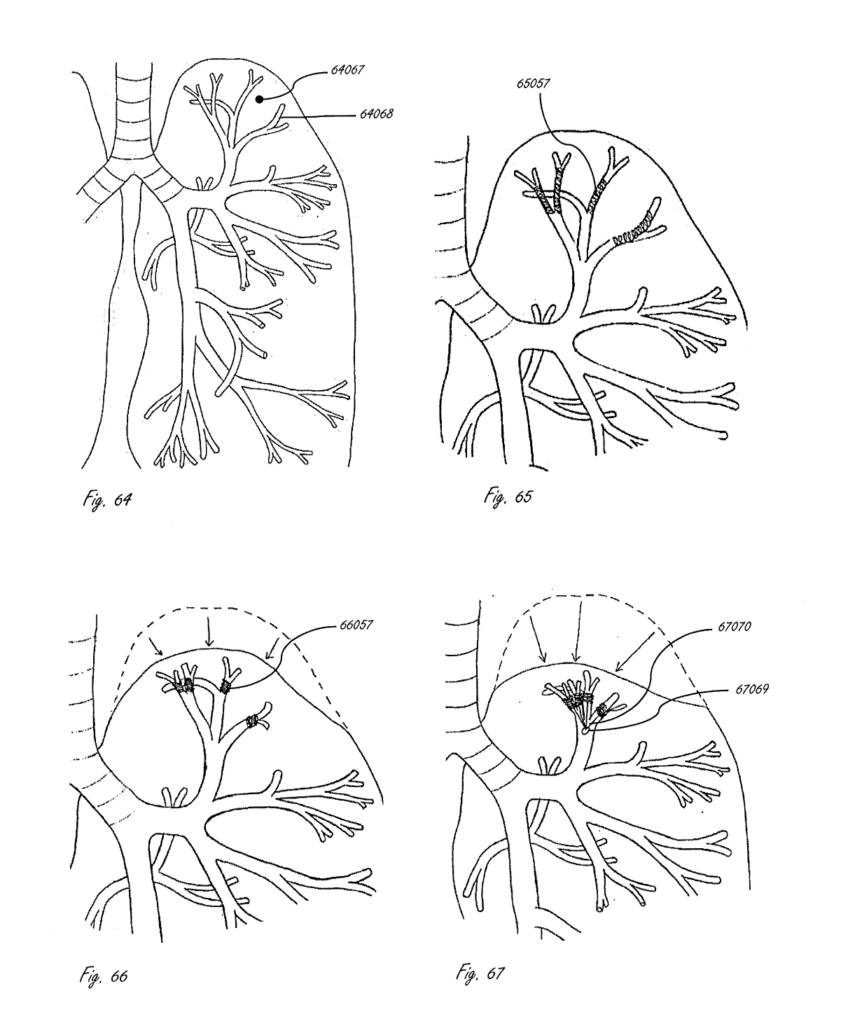

FIGS. 59 and 60 illustrate exemplary ways to increase the extent of tissue engagement and improve reduction. In FIG. 59 a plurality of sharp tines (59061) run along the trailing edge of the longitudinal element. By incorporating this feature into either a helical or torsional spring design the tissue would be less apt to slip or become disengaged from the surface of that element as the device length shortens. FIG. 60 illustrates how this tine feature can be incorporated into a raised element (59059) within the face of the element. One potential means for deploying or raising this feature would be to include a release wire (59060) within the design. The raised feature could be shape-set in a deployed, expanded, state, and then held flat until desired via the wire which would be woven over each raised feature and under the main structure of the longitudinal element. By withdrawing this wire the tines would be allowed to raise and become proud. If the procedure dictates this be done prior to releasing the stored spring force of the device, the tines could further ensure tissue engagement as the elements draw together. This release wire could also be used in the design illustrated in FIG. 59 to control the width or length of the longitudinal elements (59059). Through controlling these items the tines could be allowed to further engage the tissue through the release of some stored spring energy within the element.

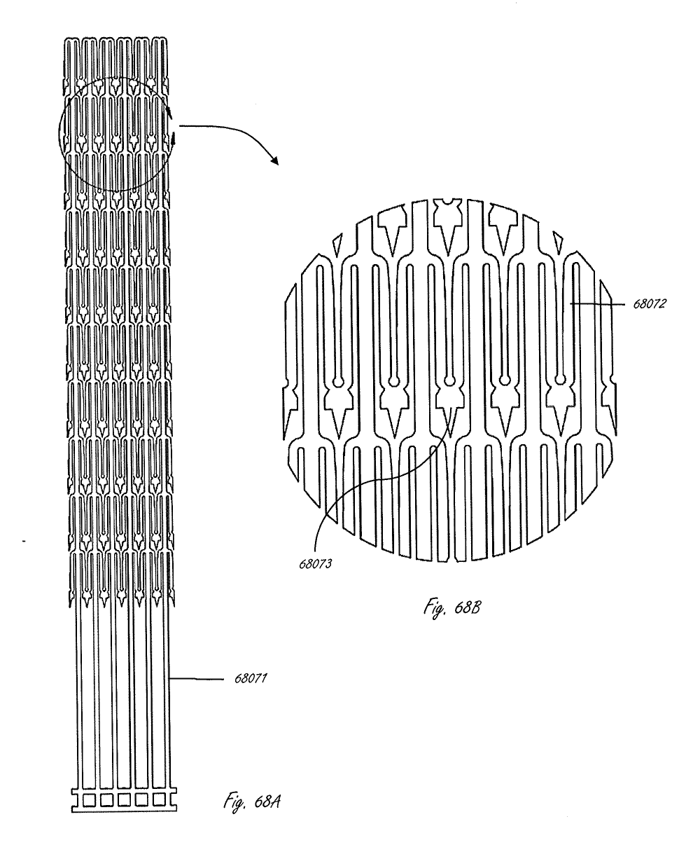

FIG. 61 illustrates an alternative embodiment of a tensioning mechanism in a treatment device. In this embodiment spool (61063) is set on a fixed diameter portion on a tube (61004) into which a stent like reconfigurable structure is cut at both ends of the tube on either side of the spool. These expanded elements keep the structure in place within the airway while a tether that runs from a distal anchor to this structure is wound up on the spool. A drive shaft is advanced to the structure and when rotated the spool also rotates which winds and unwinds the tether (61004) depending on the direction of rotation. A ratcheting feature is integrated into the spool and tube interface to prevent any undesired rotation of the spool. This design allows for a desired tension to be applied to the tether and maintained.

FIG. 62 illustrates an alternative embodiment of a tensioning mechanism in a treatment device. In this embodiment a stent like structure is cut into a tube (62064) which is then inverted. It is onto this inverted portion of the tube that a spool (62063) is mounted. A drive shaft is then advanced to the structure where it interfaces with the core rod of the spool (62065). Any rotation of the drive shaft is transferred to the spool which winds or unwinds the tether (62004) depending on direction of rotation. A ratcheting feature is integrated into the spool and tube interface to prevent any undesired rotation of the spool. This design allows for a desired tension to be applied to the tether and maintained.

FIG. 63 shows another embodiment of a tensioning mechanism. This design is a slight variation of that show in FIG. 62. This design also utilizes a spool (63063) mounted on a laser cut tube (63066) that is used to hold and adjust tension by winding and unwinding the tether (63004). The variation for this design is that the stent like structure cut into the tube is only incorporated into one end of the tube. The stent-like structure in this embodiment has a tapered proximal end, and as such is configured to be collapsed and retrieved into a retrieval catheter upon engagement between the tapered end and the retrieval catheter (a retrieval catheter could be advanced or the stent-like structure could be proximally withdrawn to initiate the collapse of the structure). The distal anchor could also be configured in such a way to allow for its collapse and retrieval subsequent to the proximal anchor retrieval. Retrieval of the proximal anchor or both anchors can be performed to reduce tension that has been applied to the lung, or return the patient towards the pre-operative states (regardless of whether the actual pre-op state is fully achieved or not). If just the proximal anchor is to be removed, the tether can be cut, leaving the distal anchor implanted.

FIGS. 64-67 describe alternative methods and devices for lung volume reduction. FIG. 64 shows an illustration of a hypothetical human lung for a patient suffering from emphysema. The hypothetical target tissue for volume reduction (64067) is identified in the left superior lobe, or upper right of the illustration. Each device is individually introduced into the desired airway (64068) of the lung. A single device (65057) can be delivered to a single airway or multiple devices to several airways, as seen in FIG. 65. The device is released, and as it foreshortens from the spring force it draws in the engaged tissue, reducing the volume of the tissue attached to the airway in that portion of the lung, as seen in FIG. 66. The devices can stand alone as a unitary feature, or can be connected to a central node (67069) at a bifurcation via anchoring lines (67070) as shown in FIG. 67. Should anchoring lines be drawn to a node a tethering system could be used to fix the lines and hold them in place. This system could allow for adjustability through the ability to individually change the tension on each of the anchoring lines. Each device is removable, as is any node or anchoring line that may be added as an option. The devices (65057) may be comprised of a super elastic material such as but not limited to memory metals. Additionally, in some configurations the elements (65057) may rely on the memory characteristics to transform from a delivery to a delivered configuration at implant. In particular as is known the device (65057) can be delivered at one temperature lower than body temperature, and rely on body heat to bring about a transition into the compressed state. Alternatively, the design can comprise a transition temperature greater then body temperature and rely on heating the device (65057) after delivery using the delivery tool, either by direct heating or joule heating mediated by inductive coupling.

FIGS. 68A and 68B illustrate an exemplary flat pattern design for a stent-like anchor that could be delivered to the periphery of the airway in the lung. The embodiment in FIGS. 68A and 68B could be delivered according to the methods in FIGS. 64-67. The anchor would be fixed to a tether attached to the proximal ring or proximal anchor (not shown). When deployed the device is capable of expanding to roughly five times its original diameter. As the device expands the distance between the longitudinal elements (68071) increases which straightens out the struts (68072). The tines (68073) would be placed along the ends of each of these struts and be configured such that either through shape-setting or mechanical interface would become proud and stick up from the expanded surface of the device. These tines would engage the tissue and help to hold the anchor in position within the airway. Through sizing and potentially shape-setting, the final diameter of the device is in excess of that of the airway it in the deployed state. As such, the device remains in contact with the tissue of the airway wall as the airway moves and the diameter fluctuates. Having affixed to the distal portion of the airway, the tethers could then be pulled to the appropriate tension for each device and the tethers gathered at a node, much like that previously shown in FIG. 67. By drawing in each anchor and pulling the airways the volume of that portion of the lung is reduced. Should adjustments need to be made the tension in each line could be adjusted (examples of which are described herein), or the tether cut all-together to release that portion of tissue. This adjustability and removability ensures that the device platform is applicable to the widest range of patient needs and procedural constraints.

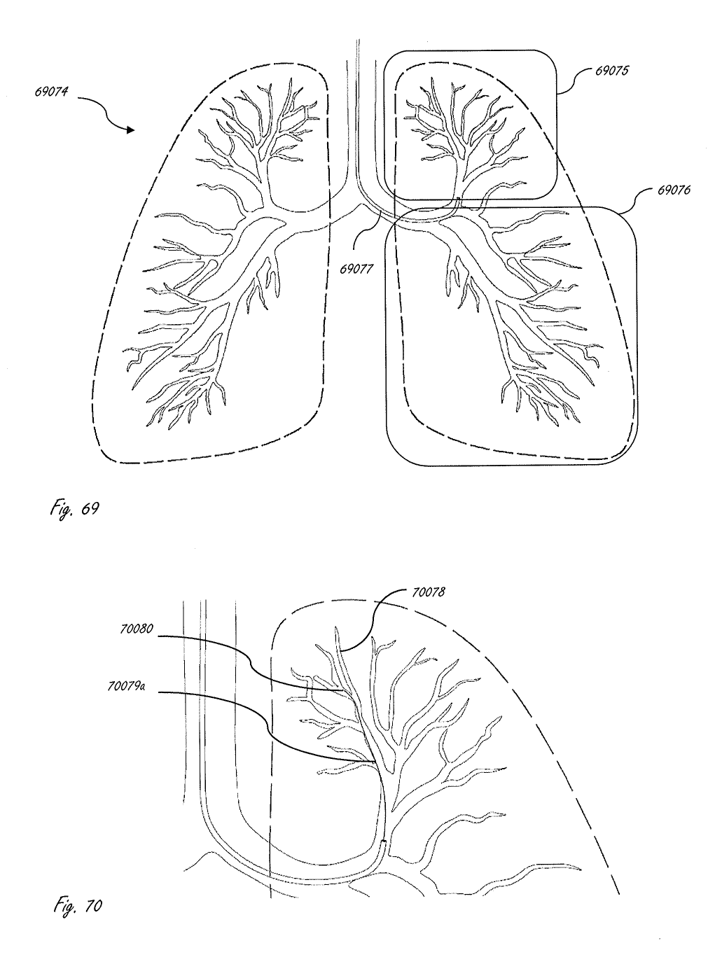

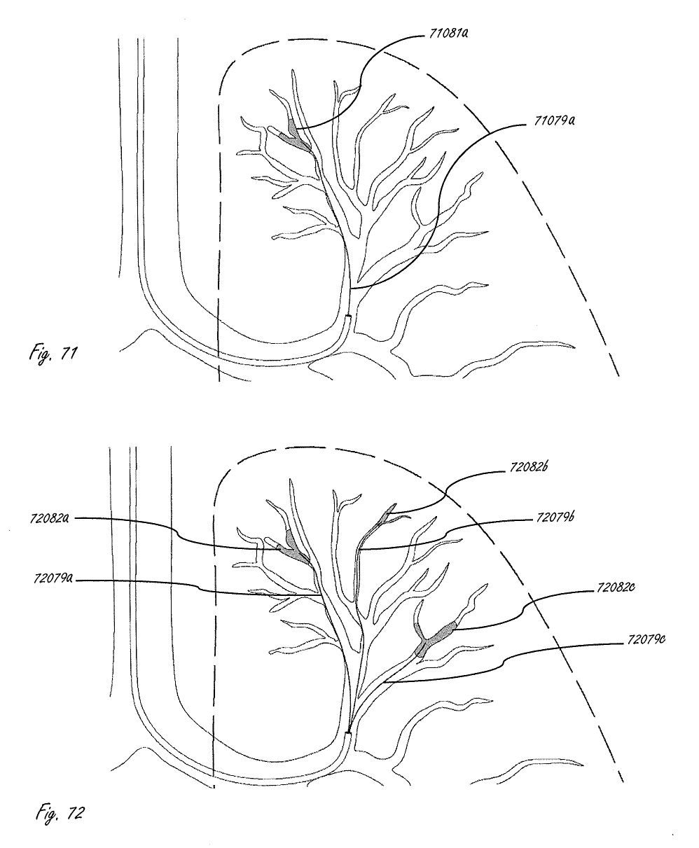

FIG. 69 illustrates a lung (69074) with a diseased upper lobe (69075). The diseased upper lobe is characterized as having a poor oxygen transport and may additionally be hyperinflated (i.e., having a larger volume than it would in a healthy state). Hyper-inflation is not shown here, but it can be appreciated that it would have a larger volume and compress the lower lobe (69076) due to the spatial constraints within the chest cavity. An endoscope (69077) has been tracked within the bronchial tree so that its tip is engaging within the upper lobe.

FIG. 70 illustrates a close up view of the diseased upper lobe of the lung, and a first step in the lung volume reduction procedure. A small diameter catheter (70079a) is advanced into a segment of distal bronchial lumen (70078). The small diameter catheter may be advanced directly, or may be advanced with the aid of a guidewire if the risk of damaging the bronchial tree is considered to be high. The tip of the small diameter catheter (70080) is placed in a segment of the bronchial tree having one or more bifurcations within its structure.

FIG. 71 shows a next step in the lung volume reduction procedure. With the small diameter catheter (71079a) placed in the diseased upper lobe, a curable material (71081a) is injected into the segment of distal bronchial lumen. The material is then cured to transform from a fluid (i.e., flowable) to a solid (i.e., non-flowable) state. Upon curing, the distal end of the catheter is bonded within and affixed to the cured material (71081a).

FIG. 72 illustrates additional small diameter catheters (72079b, 72079c) positioned within the lung, and curable material (72081b, 72081c) injected and cured to affix the catheters within the material as described in FIG. 71.

FIG. 73 shows that once the small diameter catheters (73079a, 73079b, 73079c) are in place and affixed to the injected curable material, an anchoring catheter (73083) is advanced as far forward (distally) as possible. The advancement of the anchoring catheter is stopped upon reaching a bifurcation (73084) where at least one of the small diameter catheters passes down a different branch of the bronchial tree than one or more other small diameter catheters.

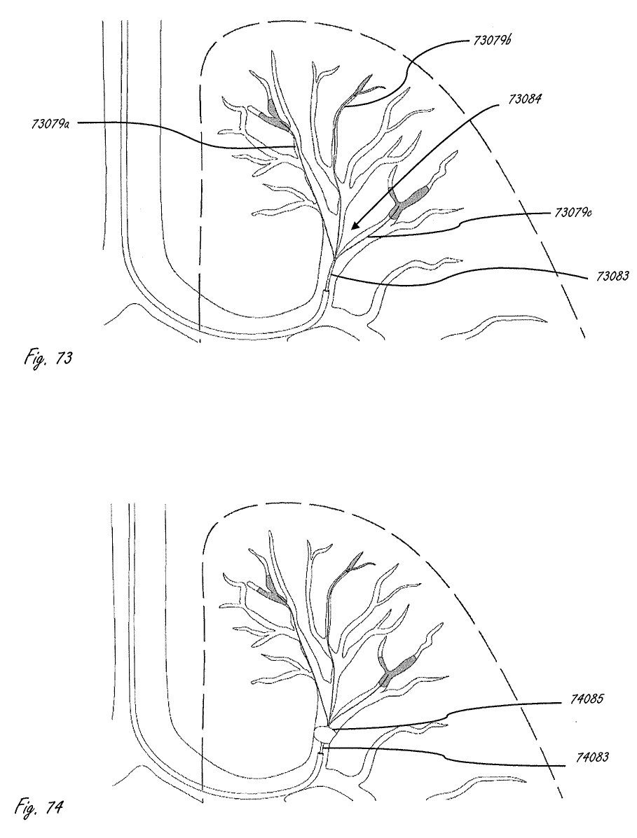

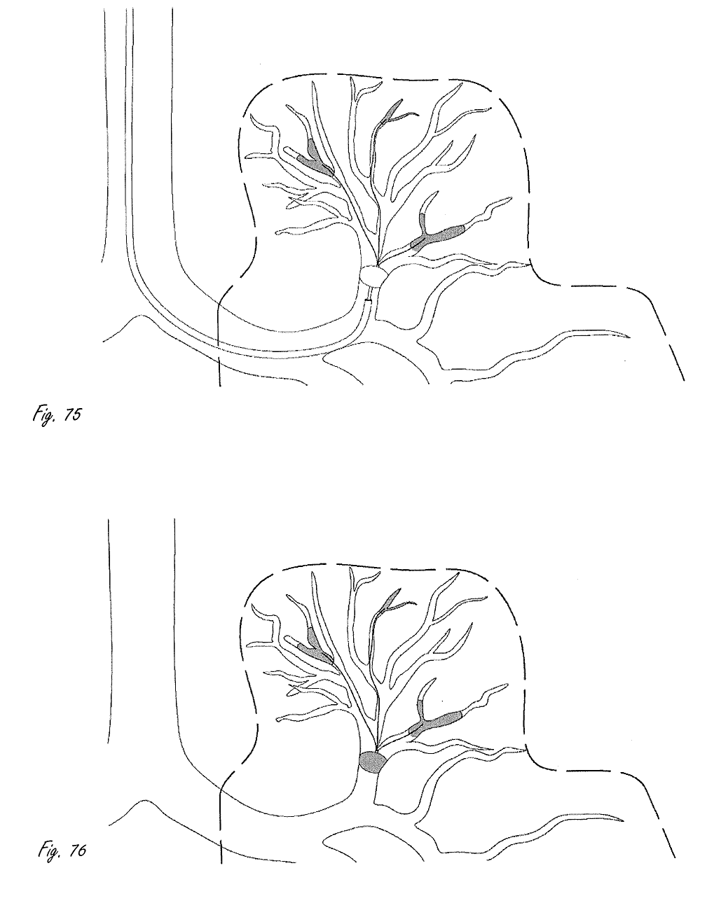

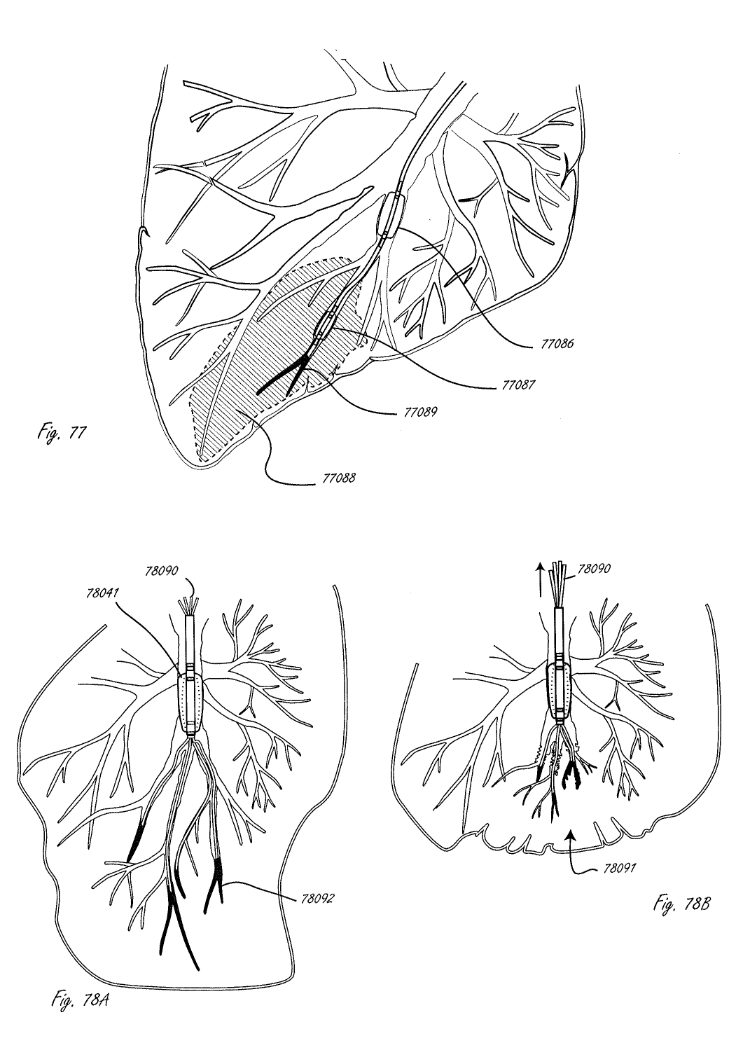

FIG. 74 shows that once the anchoring catheter (74083) is in place, an anchor (74085) can be deployed to stabilize the anchoring catheter in the bronchial tree. In this case, the anchor (74085) is shown as a balloon that has been inflated with a curable material so that it cannot deflate once the material has cured. Alternatively, the balloon could be inflated with a more common fluid (e.g., saline, air) and the pressure maintained via a one-way valve in the inflation lumen.

FIG. 75 illustrates that with the proximal anchor deployed, the small diameter catheters are retracted back into the anchoring catheter. The applied tension is transmitted through the small diameter catheters into the curable material injected into the distal bronchi, and into the lung tissue itself. Because the curable material has formed the shape of the distal bronchi, including one or more bifurcations, it cannot slip within the bronchi, but must displace the lung tissue centrally towards the anchoring catheter. As a result of the displacement of lung tissue toward the centrally located anchor catheter, the lung volume is effectively reduced.

FIG. 76 shows that once lung volume reduction has occurred, the small diameter catheters are trimmed at the anchor, and the anchor is detached from the anchoring catheter. The small diameter catheters are locked within the anchor, preventing the lung tissue from expanding back into its previous hyper-inflated condition.

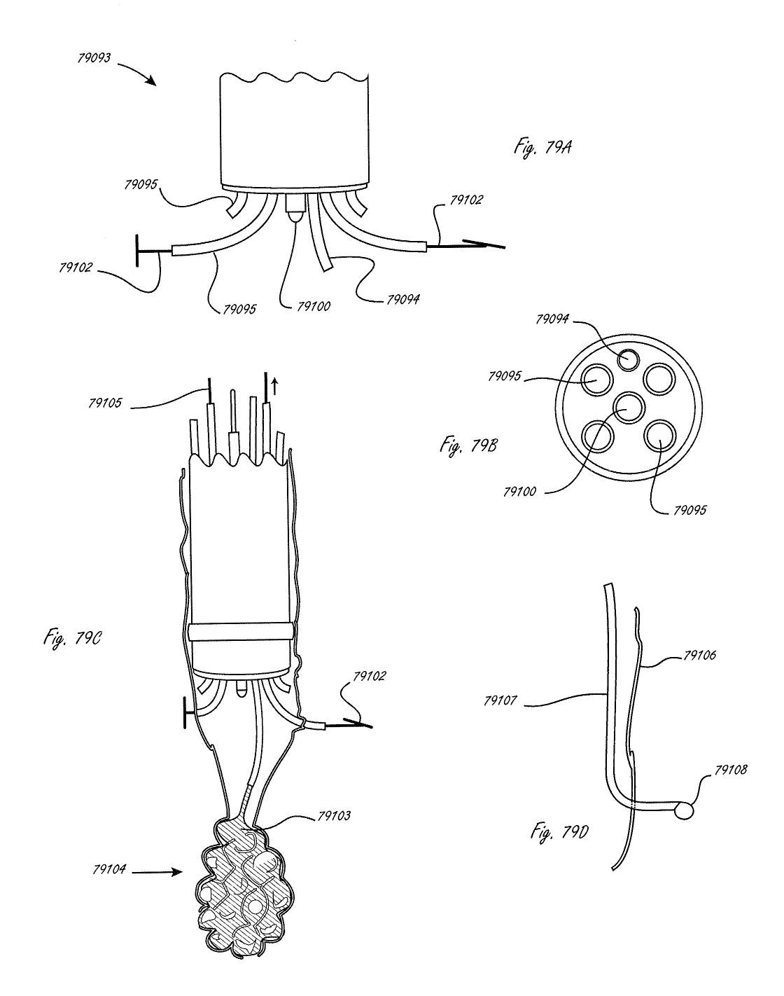

FIG. 77 illustrates an alternate embodiment in which a primary anchor balloon (77086) is deployed with a retractable distal secondary balloon (77087) to a target location. Once primary anchor balloon (77086) is positioned, secondary balloon (77087) is advanced within an airway to a diseased area of the lung (77088). Extending through the delivery catheter is a lumen capable of delivering an adhesive to the distal end of the balloon assembly. Upon placement of balloon (77087) an adhesive is delivered (77089) to seal the airway and secure the distal balloon in place. After adhesive curing, balloon (77087) is pulled upward toward primary balloon (77086) thereby compressing the airway below.

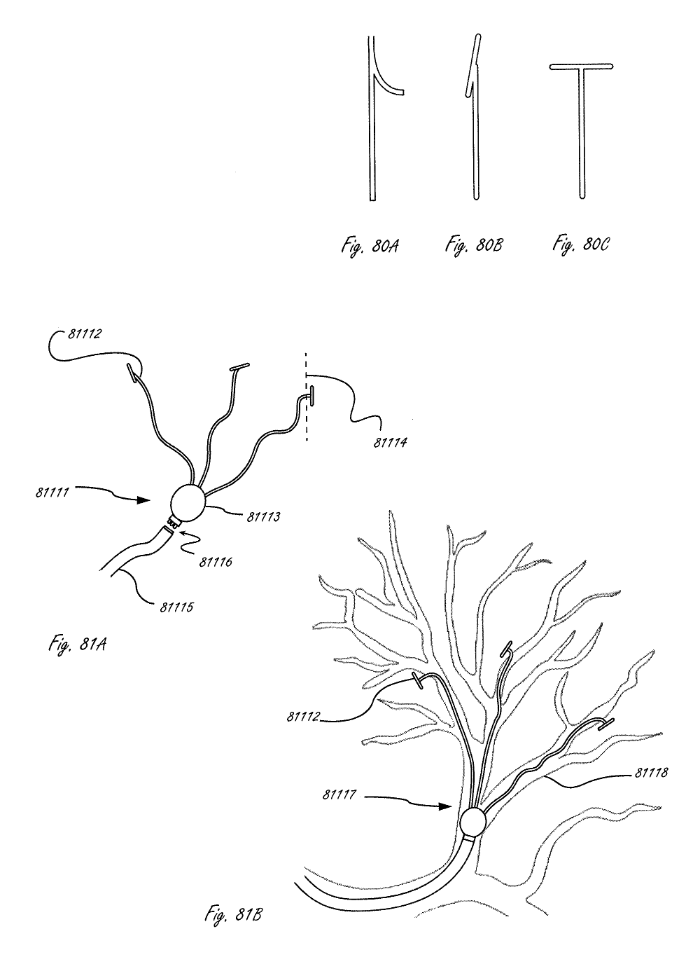

FIGS. 78A and 78B illustrate an additional exemplary method of lung volume reduction. An anchor balloon (78041) is anchored in an airway. Extending through the delivery catheter are a plurality of flexible tubes (78090) adapted to deliver an adhesive. The adhesive tubes are extended distally from the anchor balloon and an adhesive is expelled into the diseased area (78091). The adhesive delivery tube may be comprised of or comprise a fiber optic material able to deliver a curing light to the adhesive area in vivo. After curing, the adhesive tubes are pulled upward bringing the bound tissue with it and compressing that portion of the target area (78092).

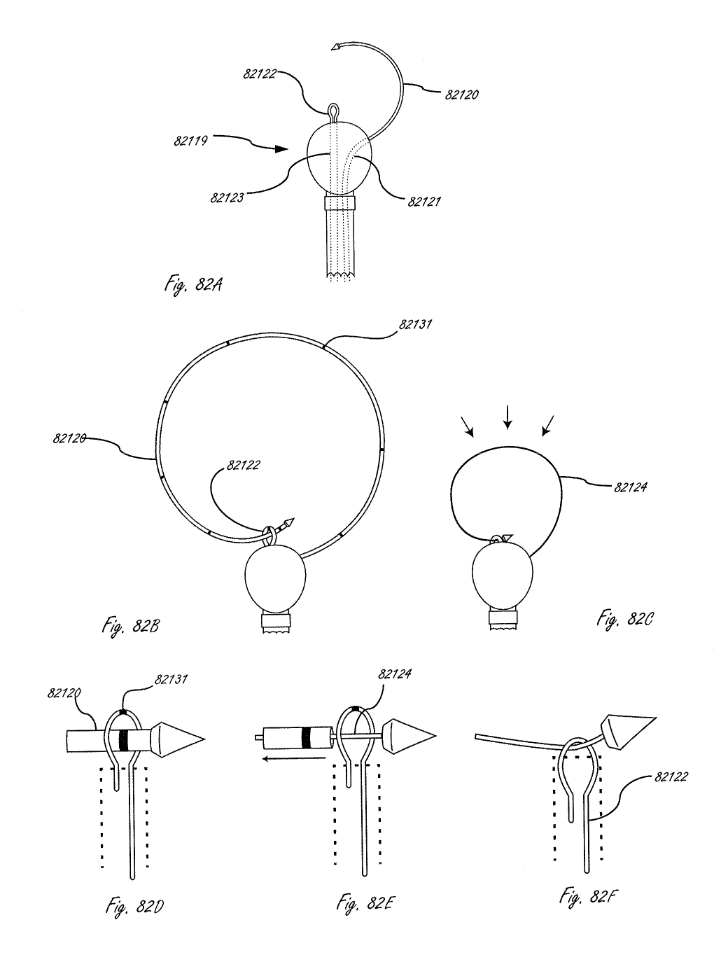

FIGS. 79A-79C illustrate an additional exemplary method of lung volume reduction. Multi-lumen delivery catheter (79093) includes adhesive delivery tubes (79094), directable hypo-tubes able to pierce an airway (79095), adhesive curing element, intra-bronchial ultrasound and/or local ventilation monitor for local evaluation, or other auxiliary devices (79100). Hypo-tubes (79095) are configured to deliver a barbed lead (79102) through the airway, the leads able to expand and anchor to the airway. Adhesive is then delivered to the area (79103) to seal diseased alveoli (79104) and airway tissue and to secure the anchors to the airway. The catheter can be used to install multiple anchor and adhesive points, pulling the anchor leads upward and compressing the targeted tissue.

FIG. 79D illustrates an outer airway wall (79106) that has been pierced by a hypo-tube or hollow tether (79107). An adhesive bleb (79108) is affixed to the outer airway wall. Adhesive bleb (79108) may be used alone as an anchor to the outer airway wall or in combination with barbed anchor (79102).

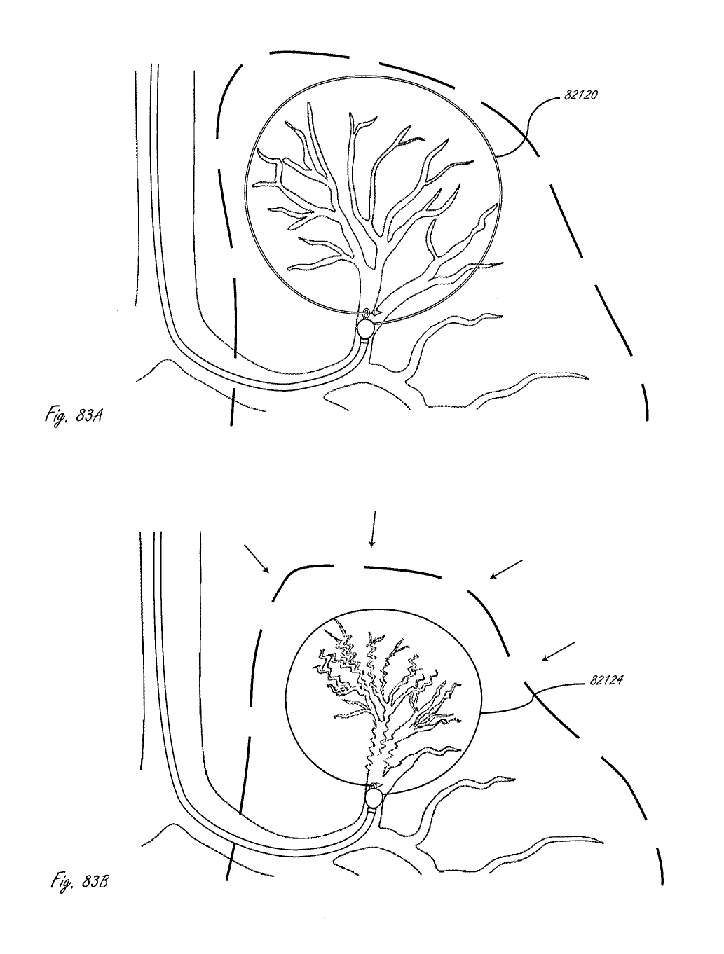

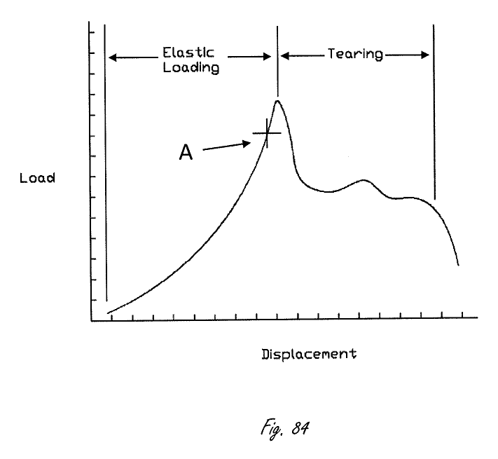

In the embodiments of FIGS. 80A-80C and 81A and 81B, a barbed lead is anchored to the outer wall of an airway. The barbed leads are then retracted back through the channeled bead compressing the surrounding lung tissue as described in other methods disclosed herein. The tethers are locked into their retracted position in the bead when their channels are deformed after crimping the bead. The delivery catheter then detaches from the crimped bead at an airway junction leaving it secured with the compressed tissue.

FIGS. 80A-C illustrate two possible styles of barbed leads. FIG. 80A depicts a fish-hook style barb; FIG. 80B depicts a “T”-style barb in its delivery position and in FIG. 80C a T-style barb is in its deployed or anchor position. Barbed leads may be made of a shape memory material enabling its complying to a delivery position during travel through a delivery lumen such as a hypotube and self-deploying to an anchor position when pushed out the tube or after the tube is removed. Such devices may be delivered through a hypotube incorporated in the delivery apparatus.

FIGS. 81A-B illustrate a “T”-style barbed lead (81112) in an anchoring system (81111) comprising a channeled bead (81113) through which a plurality of barbed leads provided with the ability to penetrate an airway wall (81114) can be extended and retracted. This channeled bead is coupled to a delivery catheter (81115). The channeled bead is configured such that the bead may be crimped forcing retracted leads to lock in a fixed position. The channeled bead also has the capability to detach (81116) from the delivery catheter. FIG. 81B shows this embodiment positioned inside an airway at an airway branch (81117) after barbed leads (81112) are anchored externally to the airway wall (81118) and prior to retracting the barbed leads.

FIGS. 82 and 83 represent another embodiment wherein lung volume reduction is achieved by extending and looping a circular shape-memory hypotube encasing an alternate barbed stitching line outward from a delivery head located within an airway and, penetrating the airway wall into the surrounding lung tissue, the conforming shape-memory hypotube returns to a capture mechanism located on the delivery head where the distal end of the stitching line may be secured. Once captured and secured, the hypotube is removed and the barbed stitching line is retracted through the delivery head compressing the gathered lung tissue. The delivery head is detached from the delivery catheter after the compression is achieved and left to hold the compressed tissue in place. The hypo tube may additionally comprise electrodes which can be used to map the encased tissue and direct the capture mechanism.

In FIG. 82A a channeled delivery head (82119) through which a shape memory hypotube (82120) may be extended or retracted through delivery channel (82121) is shown. Capture mechanism (82122) may be extended and retracted through a second delivery channel (82123) in the channeled delivery head. In FIG. 82B shape memory hypotube (82120) loops around and allowing 82124 to be captured by capture mechanism (82122). FIG. 82C shows a retracted barbed stitching line (82124) secured to the capture mechanism after the hypotube has been retracted. As illustrated optional electrodes (82131) are comprised on hypotube (82120) and capture mechanism (82122). These electrodes allow for both electrical impedance mapping of the surrounded tissue and provide feedback to the user relative to the proximity of the capture mechanism to the hypotube during capture. Such information can be used to facilitate the user steering the capture mechanism during the capture process.

FIGS. 82D-F detail the hypotube and stitching line capture process. In FIG. 82D hypotube (82120) is captured. In FIG. 82E hypotube is removed and barbed stitching line (82124) is exposed. In FIG. 82F the barbed stitching line is secured to the delivery head when the capture mechanism (82122) is retracted into the head.

In FIG. 83A shape memory hypotube (82120) is extended though an airway wall and into surrounding tissue looping back to be captured by the capture mechanism. In FIG. 83B the hypotube has been removed and the secured stitching line (82124) retracted through the delivery head compressing the surrounding lung tissue achieving a reduction in lung volume. Should it be desired to remove the anchoring system, the stitching line 82124 can be clipped and the entire anchoring system removed. This can be performed, for example, if it is desired to reduce tension that has been applied to the lung.

FIG. 84 shows a graph of the relationship between load applied to a distal anchor and the resulting displacement of that anchor. A first region of the graph is labeled “Elastic Loading,” and is characterized by a monotonic increase in displacement as additional load is applied. This indicates the tissue surrounding the airway maintains mechanical integrity. A second region of the graph is labeled “Tearing,” and is characterized by an overall drop and or non-monotonic increase in the load with increasing displacement. This drop in load is due to tearing in the airway and or the surrounding lung tissue in which the anchor is placed. This tearing reduces or completely eliminates the mechanical connection between the anchor and the lung tissue targeted for lung volume reduction. The inability to apply traction to the extremities of the area targeted for lung volume reduction due to tearing limits or reduces the overall effectiveness of the lung volume reduction procedure. FIG. 84 also shows an exemplary point “A” that demonstrates the load that should be applied for optimal lung volume reduction. This point is near the maximum load that can be applied without tearing, but avoids the actual maximum to ensure no tearing occurs. One way of identifying an appropriate load point A is by correlating it with a maximum modulus value. In such procedures the tissue is retracted to a point close to but less than the predetermined maximum modulus value.

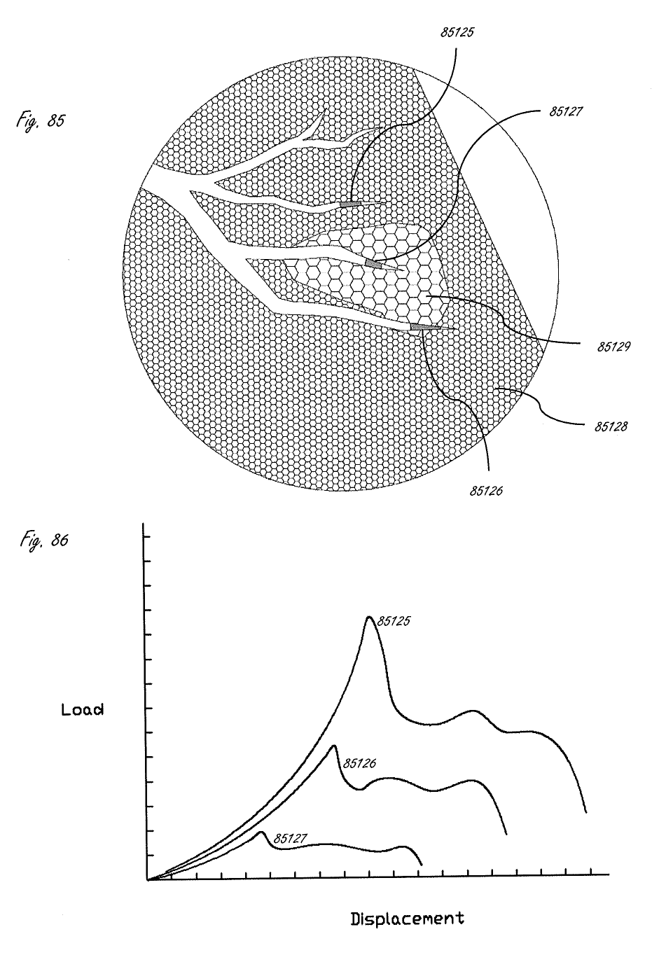

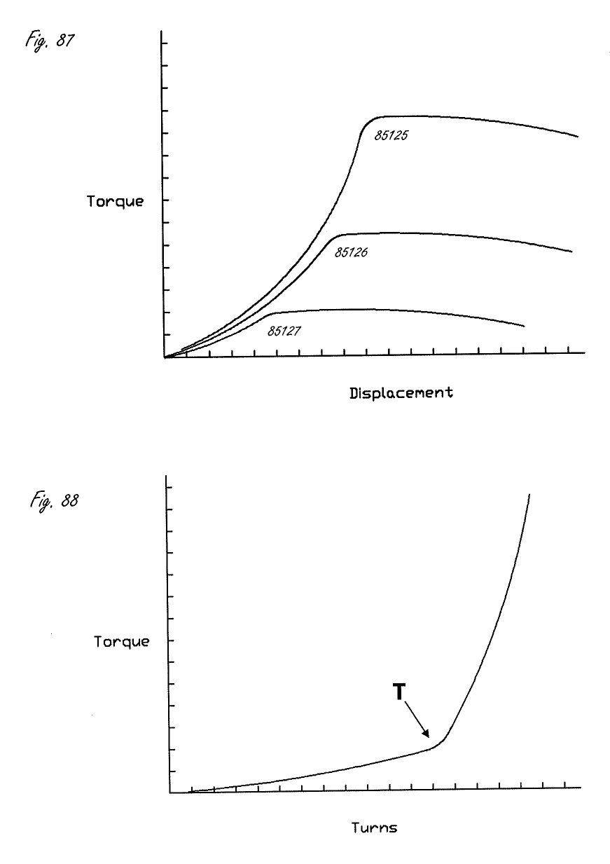

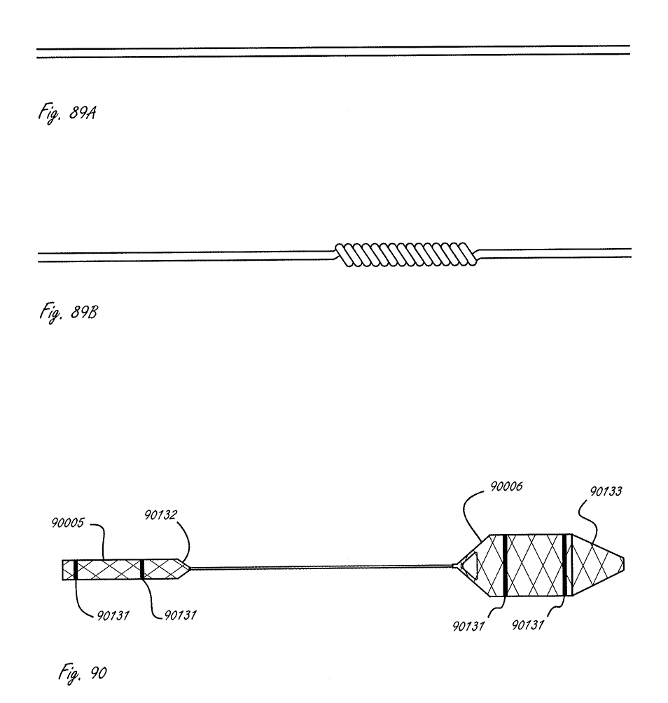

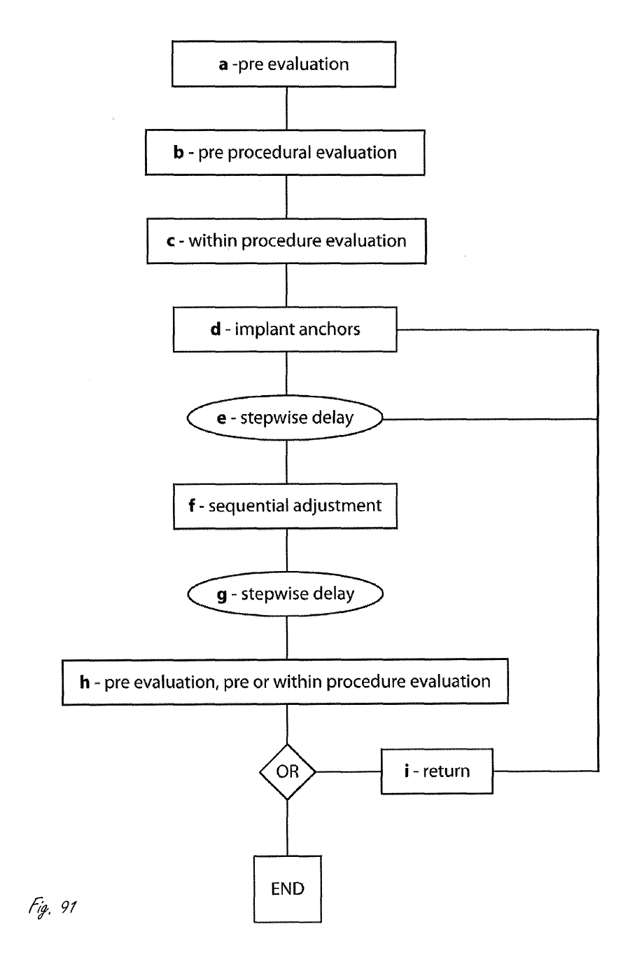

FIG. 85 and FIG. 86 show the impact of emphysematous tissue on the relationship between load applied and the resulting displacement of an anchor. In FIG. 85, anchor (85125) can be seen to reside in healthy tissue (85128), anchor (85126) can be seen to reside on the margin between healthy tissue (85128) and emphysematous tissue (85129), and anchor (85127) can be seen to reside completely within emphysematous tissue (85129). Referring to FIG. 86, anchor (85125) can be seen as achieving the highest load prior to the initiation of tearing as a result of it being placed in healthy tissue. Anchor (85126) has a lower load prior to tearing because it is engaged with both healthy and emphysematous tissue. Anchor (85127) has a very low load prior to tearing because it is only engaged with emphysematous tissue, where tearing occurs easily. The relationship depicted in the graph of FIG. 86 illustrates that the maximum load supportable can be derived from the modulus at a low displacement. In particular, the more unhealthy tissue contributing to the load deformation characteristics measured for a given anchor, the lower both the average modulus and the modulus measured at a particular displacement or load, as measured in the elastic region for that anchor. Hence one method to predetermine an appropriate load point A is to use the modulus at relatively low and safe load or displacement point to predict the target displacement or load point A.

In other alternate procedures an anchor may be displaced or loaded to the point where the modulus ceases to monotonically increase, or begins to decrease, indicating the beginning of tissue failure. In some alternative procedures, the patient can be lowed to heal at this point for a period of time as described elsewhere herein, and then the anchor can be further tightened after that healing period.

FIG. 87 shows the relationship between torque applied to the tethers attached to anchors (85125, 85126, and 85127) described in FIG. 85. Torque initially increased with displacement to a maximum where tearing occurs. Once tearing occurs, the torque stays constant, or is reduced due to the additional degrees of freedom caused by the tears.

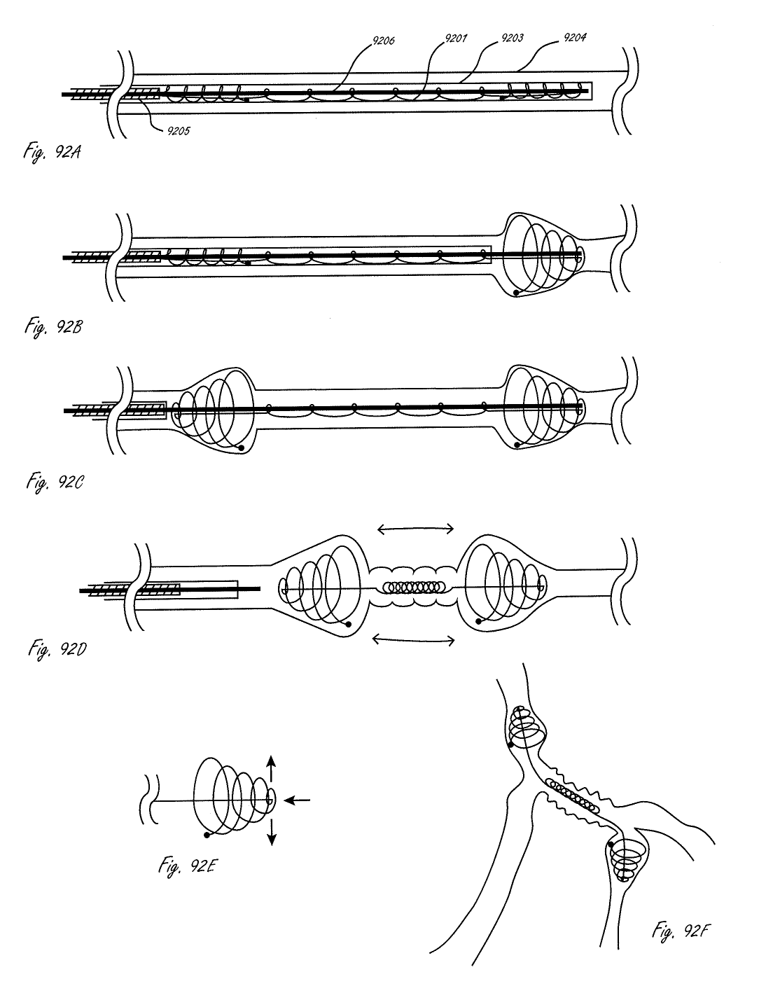

FIG. 88 shows the torque applied in a tether attached between anchors as a function of the number of turns applied to that anchor. Initially, each turn causes only a modest increase in torque. Once a sufficient number of turns has been accumulated in the line, no additional turns can be stored elastically in the line and the line forms a loop and begins to wrap upon itself. This point is shown in FIG. 88 as the point denoted as “T”. FIG. 89A shows the line before it has reached its limit in number of turns before forming a loop (point “T”). FIG. 89B shows the line having loops in it after the line has increased past point “T”. Of particular interest is that in order to cause substantial displacement of the anchors relative to each other, the torque and number of turns should exceed the critical point “T”, where the slope of the line increases substantially.

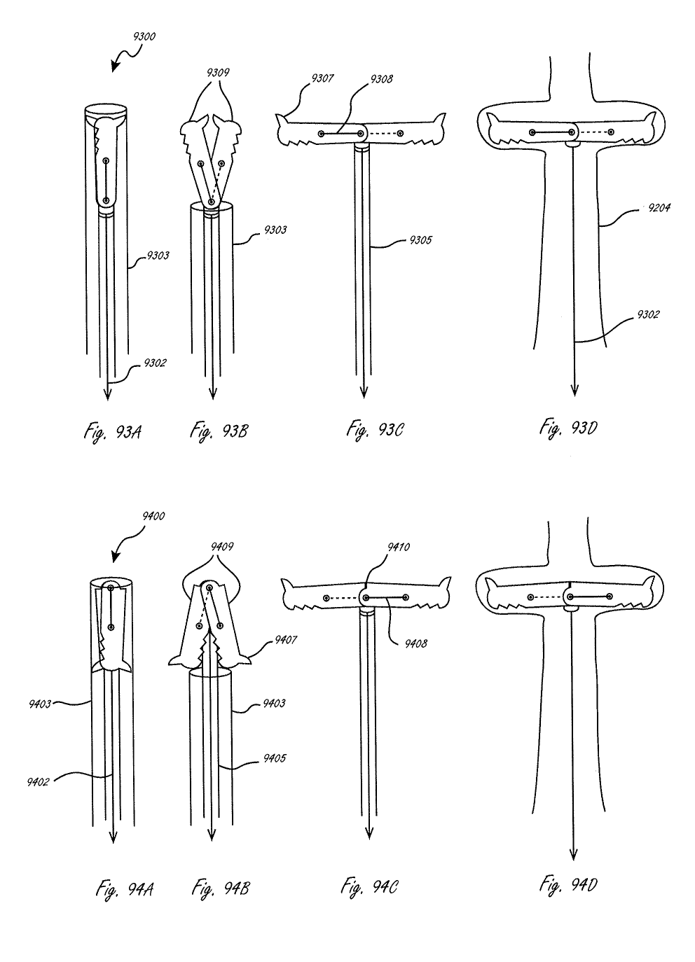

FIG. 90 is an alternative to the embodiment in FIG. 1A. FIG. 90 illustrates optional electrodes (90131) on one or both anchors. The electrodes can be used as described herein to allow for electrical impedance (EI) measurements as a way of characterizing tissue electrical impedance. In these embodiments the one or more tethers comprise a conductive element to electrically communicate with the electrodes. In other embodiments electrical impedance changes between multiple anchors may be used to indicate appropriate compression or tearing of tissues between the multiple anchors. In some embodiments the electrodes can be used to measure tissue density at the anchor and measure changes between the anchors as the anchors are drawn closer to one another. The anchoring system of FIG. 90 additionally provides a conical proximal portion (90132) on the distal anchor (90005) and a proximal conical section (90133) on the self expanding proximal anchor (90006), which facilitates complete collapse, retrieval, and removal of the anchoring system should it be desired. Either the proximal anchor or both anchors can be removed if, for example, it is desired to reduce a tension that has been applied to the lung, or return the lung towards its pre-op state.

FIG. 91 presents an exemplary flow chart of possible steps for use in performing a lung reduction volume method, examples of which are described herein. Not all steps need to be performed, and the order or steps can be modified if desired. A pre-evaluation step comprising imaging and or functional tests as described above is performed. Target and/or probable target tissues are identified at this stage. Next, a pre-procedure evaluation may be performed using minimally invasive techniques such as intra-bronchial ultrasound, local intra bronchial ventilation measurements, other characterizations of tissue density or compliance, or any pre-evaluation technique. The next step is to implant the anchors. At this point an optional stepwise delay may be initiated to allow for a healing response, tissue relaxation, and/or ingrowth. Next, a sequential adjustment is performed. This can be followed with a repeat evaluation chosen from any or any combination of those previously described. At this point additional anchors may be desired and the procedure is re-entered at step “d,” an additional stepwise delay may be initiated and the procedure re-entered at step “e,” or the procedure may be considered complete.

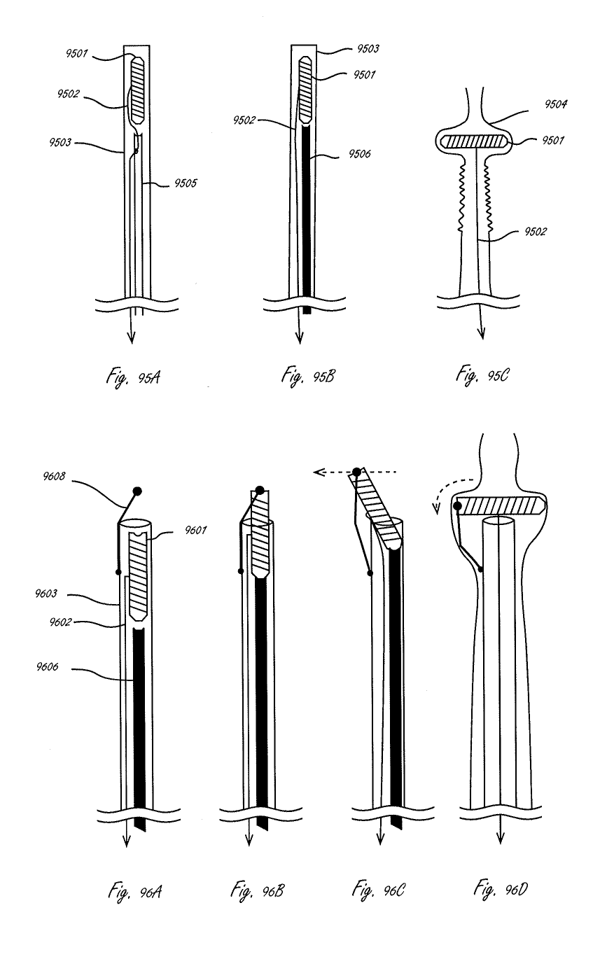

The examples shown and described with respect to FIGS. 92A-100E below build on the disclosure above, and any aspect of the systems and methods above can be incorporated into any of the examples in FIGS. 92A-100E unless indicated to the contrary.

FIGS. 92A through 100E illustrates some additional non-traumatic anchors for lung volume systems, devices, and methods of use. “Non-traumatic” as used in this context refers generally to anchors that are designed, adapted, and configured such that no portion breaks through the bronchi with which it contacts. Such devices are adapted and configured to anchor by significantly expanding the bronchi, generally by at least 50% up to 500% or 1000% while not rupturing the bronchi.

FIGS. 1 through 91 illustrate a number of traumatic and non-traumatic anchoring structures, the non-traumatic anchors primarily comprised of expanding tubular structures. FIGS. 92 through 100E illustrate alternate anchoring structures and means for use in their application of lung volume reduction.

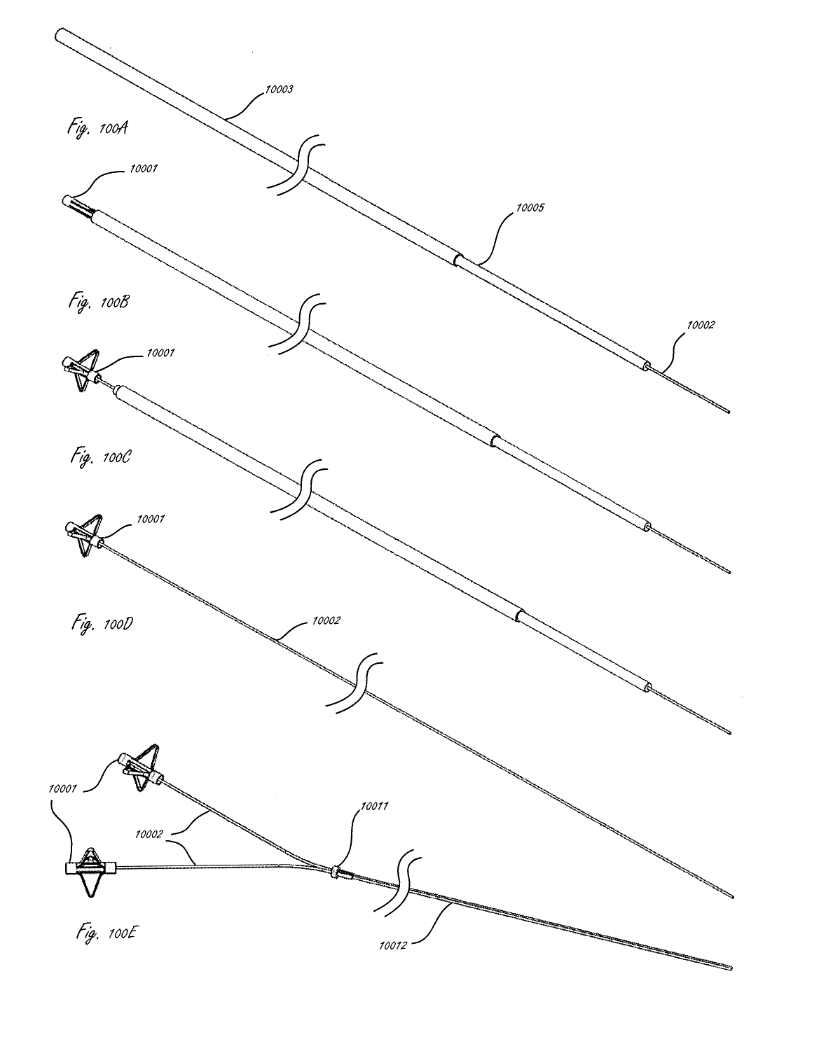

FIGS. 92A-92F depict an embodiment and deployment sequence for an anchor similar to that described in FIG. 55. In this embodiment the spring element is wound such that the conical end sections have a relaxed configuration in which the apex of the conical section is at the longitudinal extreme of the spring. Such an embodiment allows for a more abrupt transition in bronchi diameter in the direction of the applied tension. In FIG. 92A the implant spring 9201 is contained within a delivery catheter 9203 in a delivery configuration. Also comprised in the delivery apparatus are a proximally attached pusher tube 9205 and a distally attached pusher rod 9206. On delivery, the pusher rod and tube are initially moved in unison thereby holding the spring in an elongate configuration. FIG. 92B illustrates the implant 9201 after the distal end has been released from the containment within the delivery catheter and the distal end has expanded into the bronchi 9204 thereby expanding bronchi. In FIG. 98C, the spring has been completely released from within the delivery catheter 9203, while the proximal to distal distance of the spring is maintained by the pusher tube and pusher rod, and proximal and distal ends have expanded into and anchored on the surrounding bronchi 9204. FIG. 92D illustrates the bronchi, axially compressed by the spring, after the distance between the distal ends of the pusher rod and tube are no longer constrained. Note that foreshortening of the spring as shown in FIG. 92D relative to FIG. 92C causes the distal expanded sections to collapse on themselves and future support the anchoring effect as noted in FIG. 92E where the arrows indicate the expansion of the anchor as an extreme end is pulled towards the center of the spring. FIG. 92F illustrates such a spring device in a delivered (foreshortened) configuration spanning a few branches of the brachial tree.

The examples in FIGS. 93 through 100 describe alternate embodiments for anchoring systems comprised of 2 or more distal anchors and a common pull wire proximal junction, where the proximal pull wire junction provides the function of a proximal anchor. The proximal anchoring function derives from the fact that the junction sits at a bifurcation of bronchi. The exemplary devices and methods of use in FIGS. 93-100 are similar to those described in FIGS. 81A and 81B.

An unexpected result of experimenting and testing was the observation that a bronchial biopsy forceps, delivered via a bronschocope, was very difficult to remove after it was opened/expanded. It was unexpectedly observed that an anchor with a similar design and/or function could act as a distal anchor and provide adequate anchoring. FIGS. 93A, and 94B and C illustrate additional embodiments for anchoring structures which open around a central pivot to expand in a scissoring fashion. These anchors are comprised of a pull wire and an expanding anchor structure comprised of scissoring jaws which optionally comprises any or any combination of the following; a catch or stop which constrains the anchor from opening past a maximum rotation, a spring to aid in the opening after release from the delivery catheter, and barb structures which aid in the opening of the anchor by allowing the outer ends of the opening structure to engage bronchial tissue and thereby limit the anchors ability to slip along the bronchi when pushed or pulled during deployment. As illustrated each is allowed to rotate through 90 degrees and forms a linear structure in the fully deployed (which may also be referred to herein as fully expanded) configuration. The delivery structures for these embodiments comprise a delivery catheter and a means to push the anchor out of the delivery catheter.

FIG. 93A illustrates an anchor and deployment system which opens by scissoring towards the delivery tube. As illustrated the anchor comprises scissoring jaws 9309, and pull wire 9302, optional barbs 9307 and spring(s) 9308. The spring in some embodiments will be capable of fully deploying the anchor and in other embodiments the spring action may be supplemented by the action of the barbs which on engagement with the tissue allow the user to push on the center of the anchor until the anchor stop is engaged to complete the opening. FIGS. 94B and 94C illustrate an anchor and deployment system which opens by scissoring away from the delivery tube. As illustrated it comprises scissoring jaws 9409 and pull wire 9402, optional barbs 9407 and spring(s) 9408. The spring in some embodiments will be capable of fully deploying the anchor. In other embodiments the spring action may be supplemented by any of the action of the barbs which on engagement with the tissue allow the user to pull on the center of the anchor via the pull wire 9402 until the anchor stop 9410 is engaged to complete the opening, and or after the anchor is pushed out of the delivery catheter the anchor may be pulled against the delivery catheter with the pull wire 9402 thereby fully opening the anchor. FIG. 94C illustrates the anchor from either 93A or 94B in a fully deployed configuration within a bronchi, illustrating the anchoring of the anchor and the reconfiguring of the bronchi by the anchor.

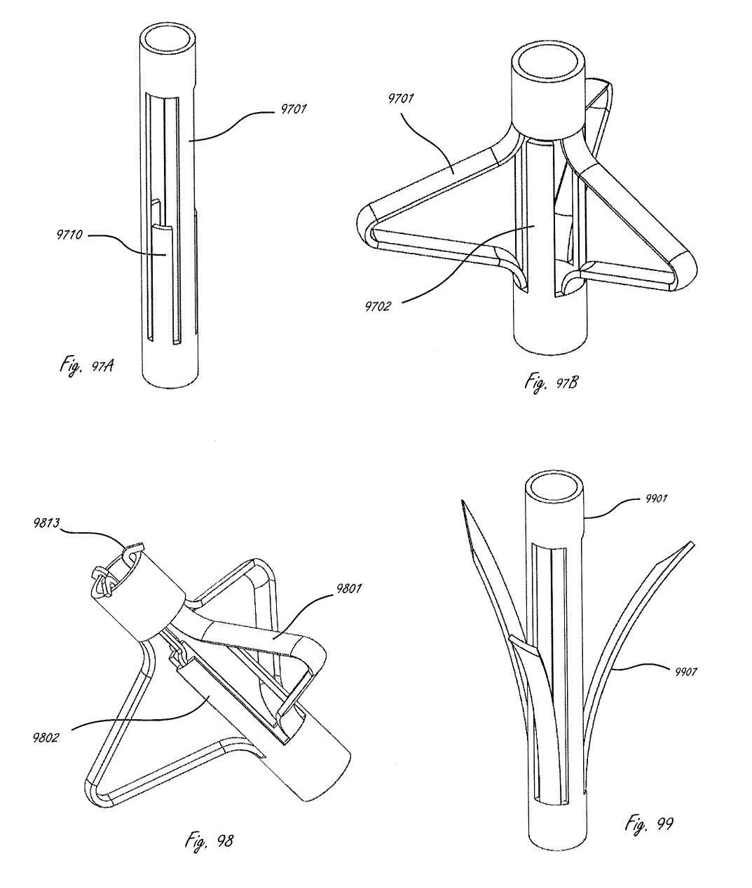

FIGS. 95A-C illustrate an exemplary non-traumatic distal anchor 9501 connected, either directly or indirectly, to a pull wire or tether 9502 that anchors by rotating 90 degrees to the bronchi axis on delivery, thereby expanding a section of the bronchi. FIGS. 95A and 95B illustrate two alternative deliver configurations for such a device. Each delivery configuration comprises a delivery catheter 9503 which may be comprised of the working channel of a bronchoscope or may be a separate catheter, an anchor 9501 attached directly or indirectly to a pull wire 9502, and a delivery tool comprising a push tube 9505 or a push rod 9506. In FIG. 95A the delivery tool push tube 9505 comprises a rapid exchange feature (which may be incorporated in any of the designs disclosed herein which use a push tube) through which the pull wire 9502 is run, in an alternate configuration the pull wire maybe run through the entirety of the push tube. Deployment is accomplished by pushing the anchor out of the delivery catheter with the push tool and then pulling the anchor back against the delivery catheter with the pull wire to force the anchor to rotate with respect to the delivery catheter. The delivery catheter is then removed, leaving the anchor in the rotated and anchored position and configuration. FIG. 95C illustrates anchor 9501 (from either FIG. 95A or 95B) in a fully deployed configuration. In such an embodiment the anchor may comprise a barb as discussed in conjunction with the embodiments illustrated in FIGS. 93 and 94 to help facilitate the rotation. FIGS. 96A-D depict an alternative delivery mechanism for an anchor similar to that of FIGS. 95A-C in which rotation of the anchor 9601 is facilitated by spring structure 9604 affixed to the side of the delivery catheter, 9603. As the anchor is pushed out of the delivery catheter the distal end of the anchor engages with the rotation spring. As the anchor is pushed further it is forced to rotate, with the distal end rotating to the left in the figures.

FIGS. 97 through 99 illustrate three additional configurations for distal anchors, two of which are non-traumatic (FIGS. 97A/B and FIG. 98) and one is traumatic (FIG. 99). Each anchor would be affixed to a pull wire attached at the distal end, which is facing the bottom of the page for each of the embodiments illustrated.

FIGS. 97A and B illustrate an expandable anchor 9701 which may be cut from a nitinol tube or similar material prior to shape setting as illustrated in FIG. 97B. The anchor can be collapsed for introduction into a deployment catheter. Anchor 9701 also comprises an optional deployment stop 9710. Such an anchor may in some embodiments be capable of full deployment as facilitated by the spring forces inherent in the shape set structure. Alternate embodiments may require additional force to deploy, such as by pushing a deployment tool against the proximal edge of the anchor while constraining any displacement of the distal end with the distally affixed pull wire. The deployment stop 9702 assures that anchor is not over-compressed in such a deployment.

FIG. 98 illustrates an anchor 9801 similar to that of FIGS. 97A-B in the shape set configuration. The anchor 9801 additionally comprises a plurality of clips 9813, cut as part of the structure, which engage the proximal end of the structure on deployment. This engagement locks the structure in a deployed configuration and elements 9802 constrain the anchor from over compression.

FIG. 99 illustrates a traumatic variation in which the barbs 9907 may be laser cut from a nitinol or similar material tube.

FIGS. 100A-E illustrate an exemplary deployment sequence and a deployed configuration for the anchor of FIGS. 97A and B.

The anchor and deployment system 10000 comprises a delivery catheter 10003, a push tube 10005, and the anchor 10001 and associated pull wire 10002. During a deployment, the deployment or delivery system is loaded into the working channel of the bronchoscope and the bronchoscope delivered to a location in visual in contact with the target location. As an alternative the bronchoscope may be delivered to the target location and then the deployment system loaded into the bronchoscope. Upon reaching a location in visual in contact with the target location the delivery system is pushed out of the distal end of the bronchoscope working channel and into the target bronchi. The push tube is then used to push the anchor out of the delivery catheter as illustrated in FIG. 100B. The anchor expands as it is released from the delivery tube. As indicated above in some embodiments the anchor may further expanded by constraining the distal end with the pull or anchor wire and pushing the proximal end with the push tube. This is especially useful when a self locking anchor such as that depicted in FIG. 98 is used. The delivery components 10003 and 10005 are then removed as illustrated in FIG. 100D. At least one additional anchor, if not more, are then placed in the general vicinity in the same fashion. Upon completion of anchor placement a clamping bead 10011 is slid down the bundled pull wires 10012. As illustrated the bundle comprises two pull wires but as indicated above such a bundle may comprise more than two pull wires 10002. The clamp 10011 is pushed until its distal end encounters at least a bronchial bifurcation into each branch of which a pull wire in the bundle trails. The clamp interfering with this bifurcation comprise the proximal anchor in such a deployment.

In some embodiments a clamp may be used in a similar fashion to gather multiple bundles of pull wires 10012, associated with already clamped and volume reduced regions, at a bifurcation more proximal than those associated with the gathered bundle to further compress lung tissue. This is especially useful when returning to further compress tissue as described in procedures elsewhere in this disclosure.

In practice multiple of such anchor placements may be used to achieve a complete LVR.

In some embodiments the delivery catheter 10003 is short and serves to load the remaining portions of the system into the working channel of a bronchoscope and the anchor deployment occurs at a location just distal to the distal end of the bronchoscope.

In some embodiments the delivery is done under flouro with the aid of an external mapping such as the CARTO or NaviStar systems without the use of a bronchoscope.

Any of the pull wires described herein, including in FIGS. 92-100, may be comprised of metals or polymers. In any of the examples the pull wire may be beaded or shaped in such a fashion that they interface with the clamp in the fashion of tie wraps.

In any of the examples in FIGS. 92-100, the width of the anchor in a deployed configuration (measured orthogonally relative to the longitudinal axis of delivery device) may be from 0.5 mm to 8 mm, and can be, for example, chosen from a kit of devices depending on the anchoring location. Some smaller lumens may need a smaller width anchor, while larger lumens may require larger dimensioned anchors for proper anchoring. Additionally, the aspect ratio of anchor width to height (height measured in the direction of the longitudinal axis of delivery device) may be 2/1 to 10/1, such as about 4/1-5/1.

Additionally, in the examples in FIGS. 93-96, the anchor is shown and described as linear. The anchor may have an expanded configuration that is not quite “T” shaped, but one that is closer to “Y” shaped. For example, in some embodiments the angle of the deployed anchor, relative to the longitudinal axis of the delivery device), can be from 90 to 135. In some embodiments it may also be slightly less than 90 degrees

This disclosure incorporates by reference herein the disclosure of U.S. Pat. No. 6,997,189 and U.S. Pat. No. 8,282,660. Any of the embodiment therein can be modified to include any of the features or methods of use described herein.

### Alternative Embodiments

Additional aspects of the disclosure are defined in accordance with the following exemplary embodiments:

1. A method for reducing the volume of a section of diseased lung comprising: identifying at least one section of diseased lung; characterizing a physical quality of the at least one diseased section of the lung; determining the location of the at least one diseased section of lung; endobronchially delivering an anchoring system to the diseased portion of the lung; the anchor system capable of, incremental adjustment to increase or decrease the distance between a proximal and distal anchor, and sustaining said adjustment upon release from a delivery system; adjusting the system to reduce the volume of the diseased tissues in the lung.

2. The method of embodiment 1 where the section of diseased lung is emphysematous comprising hyperinflated tissue.

3. The method of embodiment 1 where quality is a measure of tissue compliance.

4. The method of embodiment 3 where tissue compliance is determined using a medical imaging means prior to the implantation procedure.

5. The method of embodiment 3 where tissue compliance is determined using an endovascularly delivered ultrasonic means during the implantation procedure.

6. The method of embodiment 1 where quality is a measure of tissue density.

7. The method of embodiment 6 where tissue density is determined using a medical imaging means prior to the implantation procedure.

8. The method of embodiment 7 where tissue density is determined using an endovascularly delivered ultrasonic means during the implantation procedure.

9. The method of embodiment 1 where the physical quality is used to determine the maximum tension to apply to a distal anchor.

10. The method of embodiment 9 where the maximum tension to the anchor is as amount of determined to sustain no or minimal parenchyma tearing in the tissue surrounding the anchor.

11. The method of embodiment 1 where location is determined via a medical imaging means prior to the implantation procedure.

12. The method of embodiment 11 where location is characterized as the tissues bounding and internal to the boundary of healthy tissue.

13. The method of embodiment 11 where delivery comprises placing one or more distal anchors within or at the boundary of diseased tissue and placing at least one or more proximal anchors within healthy tissue or at the boundary of the healthy tissue.

14. The method of embodiment 11 where delivery comprises placing one or more distal anchors within diseased tissue and placing at least one or more proximal anchors within diseased tissue or at the boundary of the healthy tissue.

15. The method of embodiment 1 actuating the proximal anchor to reduce the volume of the diseased section of lung.

16. The method of embodiment 15 where actuating reduces the distance between a proximal and distal anchor.

17. The method of embodiment 15 where actuating causes the tether to wind on itself.

18. The method of embodiment 17 delivering multiple anchor systems to a single diseased section of lung.

19. The method of embodiment 1 delivering one or more anchor systems to multiple diseased sections of lung.

20. The method of embodiment 1 where the magnitude of the tension on an anchor is determinable in situ by a medical imaging means.

21. The method of embodiment 1 where the any distal anchor may be released from a proximal anchor.

23. A method for reducing the volume of a section of diseased lung comprising: endobronchially delivering an anchoring system to a diseased portion of the lung where the system is comprised of at least one proximal anchor and at least one distal anchor; the anchor system capable of incremental adjustment to increase or decrease the distance between a proximal and distal anchor, and sustaining said adjustment upon release from a delivery system; actuating the system to reduce the volume of the diseased tissues in the lung; allowing a period of time to pass and then readjusting the distance between the at least one proximal anchor and at least one distal anchors.

24. The method of embodiment 24 the period of time sufficient to allow for any or any combination of the following: tissue relaxation; tissue ingrowth into the anchors; healing response in the volume reduced tissue.

25. The method of embodiment 24 the period of time in the range of 5 minutes to greater than 1 year.

26. The method of embodiment 24 identifying at least one section of diseased lung prior to delivery of the anchoring system.

27. The method of embodiment 24 characterizing a physical quality of the at least a portion of one diseased section of the lung.

28. The method of embodiment 24 determining the location of the at least one diseased section of lung.

29. The method of embodiment 24 the anchoring system comprising multiple distal anchors

30. The method of embodiment 24 delivering multiple anchor systems to a single diseased section of lung.

31. The method of embodiment 24 delivering anchor systems to multiple diseased sections of lung.

33. The method of embodiment 24 actuating is adjusting the distance between at least a proximal and distal anchor.

34. The method of embodiment 24 perform any preplaning or in situ study between adjustments.

35. The method of embodiment 24 to prevent pneumothorax.

36. The method of embodiment 24 where the magnitude of the tension on an anchor is determinable in situ by a medical imaging means.

37. A method for reducing the volume of a section of diseased lung comprising: identifying at least one section of diseased lung using an endobronchial ultrasound device to determine a physical quality of the lung tissue in the at or near the diseased tissue; endobronchially delivering an anchoring system to a diseased portion of the lung where the anchor system comprising at least one proximal anchor and at least one distal anchor; the system capable of incremental adjustment to increase or decrease the distance between a proximal and distal anchor, and sustaining said adjustment upon release from a delivery system; adjusting the system to reduce the volume of the diseased tissues in the lung;

38. The method of embodiment 37 where the physical quality is compliance

39. The method of embodiment 37 where the physical quality is a measure of the loading capacity an anchor.

40. The method of embodiment 37 where the loading capacity of the anchor is a measure of the amount of load the anchor can sustain without parenchyma tearing.

41. A method for reducing the volume of a section of diseased lung comprising: endobronchially delivering an anchoring system to the diseased portion of the lung where the system comprises at least one proximal anchor and at least one distal anchor, the anchor system capable of incremental adjustment to increase or decrease the distance between a proximal and distal anchor, and sustaining said adjustment upon release from a delivery system; and adjusting the system to reduce the volume of the diseased tissues in the lung.

42. The method of embodiment 41 where the system is adjusted to a predetermined tension on the anchor.

43. The method of embodiment 41 where the predetermined tension is characterized by any one or combination of the following: medical imaging system, endobronchial US, a local functional measurement.

44. The method of embodiment 41 adjusting is reducing or increasing the distance between at least a proximal and distal anchor.

45, The method of embodiment 41 incrementally adjusting multiple distal anchors such that the tension on each distal anchor never exceeds a predetermined value.

46. The method of embodiment 45 incrementally adjusting anchors in less diseased tissue prior to adjusting anchors in more diseased tissues.

47. The method of embodiment 45 comprising simultaneous treatment of an entire lung by incrementally adjusting multiple distal anchors connected to multiple proximal anchors

48. A method for reducing the volume of a section of diseased lung comprising: endobronchially delivering a lung volume reduction system to a portion of the lung; the lung volume reduction system comprised of an anchoring system comprising at least one proximal anchor, at least one distal anchor, and a means for monitoring a ventilation parameter at a target bronchi or bronchiole; the anchor system capable of incremental adjustment to increase or decrease the distance between a proximal and distal anchor; and sustaining said adjustment upon release from a delivery system; determining from the monitored ventilation parameters an implant location for a proximal anchor; adjusting the system to reduce the volume of the diseased tissues in the lung.

49. A method for reducing the volume of a section of diseased lung comprising: endobronchially delivering an anchoring system to the diseased portion of the lung where the anchor system comprises at least one proximal anchor and at least one distal anchor; the anchor system capable of incremental adjustment to increase or decrease the distance between a proximal and distal anchor, and sustaining said adjustment upon release from a delivery system; adjusting the system to reduce the volume of the diseased tissues in the lung.

50. The method of embodiment 49 where the system is incrementally adjusted to a predetermined tension on the anchor.

51. The method of embodiment 50 where the predetermined tension is characterized by any one or combination of the following: medical imaging system; endobronchial US; and a local functional measurement.

52. The method of embodiment 50 adjusting is reducing or increasing the distance between at least a proximal and distal anchor.

53. The method of embodiment 50 incrementally adjusting multiple distal anchors such that the tension on each distal anchor never exceeds a predetermined value.

54. The method of embodiment 53 incrementally adjusting anchors in less diseased tissue prior to adjusting anchors in more diseased tissues.

55. The method of embodiment 53 simultaneous treatment of an entire lung by incrementally adjusting multiple distal anchors connected to multiple proximal anchors.

### Alternative Embodiments

Additional aspects of the disclosure are defined in accordance with the following exemplary embodiments:

56. A device for reducing the volume of a lung, comprising: a distal anchor, a proximal anchor, and a tether extending between the distal and proximal anchors, the device configured so that the distance between the anchors measured along the tether can be increased or decreased and maintained after release of a delivery device.

57. The device of embodiment 56 wherein the device is further configured so that the distance between the anchors can be further increased or decreased after the device has been released from a delivery device.

58. The device of any of embodiments 56-57 wherein the device further comprises a tensioning controller that interfaces with the tether, the tensioning controller configured to be actuated to increase or decrease the distance between the proximal and distal anchors.

59. The device of any of embodiments 56-58 wherein a tether actual length between the anchors stays the same.

60. The device of any of embodiments 56-59 wherein the tether is adapted to be reconfigured such that the distance measured along the tether between the anchors can be reduced.

61. The device of any of embodiments 56-60 wherein only a portion of the tether is configured to be reconfigured.

62. The device of any of embodiments 56-61 wherein the tether is configured to wind up on itself to decrease the distance between the anchors.

63. The device of any of embodiments 56-62 wherein the distal anchor is disposed at a distal end of the device, the proximal anchor disposed at a proximal end of the device, and the device does not include any other anchors disposed between the distal and proximal anchors.

64. The device of any of embodiments 56-63 wherein the distal and proximal anchors are expandable.

65. The device of any of embodiments 56-64 wherein at least one of the distal and proximal anchors has an electrode thereon.

66. The device of any of embodiments 56-65 wherein the device is configured so that as the distance between anchors changes, a tether axis remains in the same direction.

67. The device of any of embodiments 56-66 wherein the axis remains in the same direction even though the tether changes configuration.

68. The device of any of embodiments 56-67 wherein the device is configured so that as the distance between anchors changes, the rotational orientation, out of a plane comprising the tether axis, of the distal anchor stays the same relative to the proximal anchor.

69. The device of any of embodiments 56-68 wherein the proximal anchor is configured to be collapsed and removed from the lung after it has been expanded towards an expanded configuration.

70. The device of any of embodiments 56-69 wherein the distal anchor is configured to be collapsed and removed from the lung after it has been expanded towards an expanded configuration.

What is claimed is:

1. A method of reducing the volume of a lung, comprising:
endobronchially positioning an anchoring device within a lung, the anchoring device comprising at least a distal anchor;
expanding at least a portion of the distal anchor to anchor the distal anchor against lung lumen tissue; and
tensioning at least a portion of the device to reduce the volume of the lung.

2. The method of claim 1, further comprising expanding at least a portion of a proximal anchor before the tensioning step.

3. The method of claim 1, wherein expanding at least a portion of the distal anchor comprises repositioning at least a portion of the distal anchor so that the portion extends further radially relative to the lumen.

4. The method of claim 3 wherein the portion includes first and second movable elements, and the expanding step includes repositioning the first and second movable elements so that each of the elements extends further radially relative to the lumen.

5. The method of claim 4 wherein repositioning the first and second movable elements comprises pivoting the first and second movable elements so that a portion extending further radially relative to the lumen.

6. The method of claim 5, wherein the first and second elements both have proximal ends and free distal ends, the distal ends further radially outward than the proximal ends, the first and second elements optionally linear or Y-shaped when expanded.

7. The method of claim 3 wherein the portion is a single linear element, the expanding step comprising repositioning the single linear element to a position orthogonal to the lumen.

8. The method of claim 3 wherein the portion includes at least one elongate element with distal and proximal ends, and a segment between the distal and proximal ends that extend further radially than the proximal and distal ends when expanded.

9. The method of claim 3 wherein repositioning comprises expanding the distal anchor into a configuration in which a distal end of the distal anchor has a smaller radial expansion than a proximal end of the distal anchor, and optionally the distal anchor has a conical configuration.

10. The method of claim 9 further comprising, prior to the tensioning step, expanding a proximal anchor into a configuration in which a proximal end of the proximal anchor has a smaller radial expansion than a distal end of the proximal anchor, and optionally the proximal anchor has a conical configuration.

11. A device for reducing the volume of a lung, the device adapted and configured to be endobronchially positioned within a lung, the device comprising at least an expandable distal anchor, the distal anchor including at least a portion adapted and configured to be expanded to anchor the distal anchor against lung lumen tissue.

12. The device of claim 11 further comprising an expandable proximal anchor.

13. The device of claim 11 wherein the portion, when expanded, extends further radially than prior to expansion.

14. The device of claim 13 wherein the portion includes first and second movable elements, the device adapted to move the first and second movable elements so that each of the elements extends further radially than prior to expansion, the first and second elements optionally adapted to pivot so that each of the elements extends further radially.

15. The device of claim 14 wherein the first and second elements both have proximal ends and free distal ends, the first and second elements adapted so that the distal ends are further radially outward than the proximal ends, optionally linear or Y-shaped, when expanded.

16. The device of claim 13 wherein the portion is a single linear element, the single linear element adapted to be moved to a position orthogonal relative to a delivery lumen.

17. The device of claim 13 wherein the portion includes at least one elongate element with distal and proximal ends, and a segment between the distal and proximal ends that extends further radially than the proximal and distal ends in an expanded configuration.
